(19) United States
(12) Patent Application Publication
(10) Pub. No.: US 2010/0233021 A1
(43) Pub. Date: Sep. 16, 2010

(54) Systems and methods to deal with health-relevant fouling or plugging depositions and growths

(76) Inventors: John W. Sliwa, Los Altos Hills, CA (US); George W. Keilman, Woodinville, WA (US); Bryan T. Oronsky, Los Altos, CA (US); Carol A. Tosaya, Los Altos Hills, CA (US); Herbert L. Berman, Palo Alto, CA (US)

Correspondence Address: David W. Collins;Intellectual Property Law, Suite 100, 512 E. Whitehouse Canyon Road, Green Valley, AZ, 85614, US

(21) Appl. No.: 12/381,423

(22) Filed: Mar. 11, 2009

(51) Int. Cl. A61L 2/10; A61L 2/03; A61L 2/025; A61N 1/32

(52) U.S. Cl. 422/20; 422/22; 422/24; 607/72

(57) ABSTRACT

Disclosed are methods of inhibiting, avoiding or destroying existing, potential or incipient biodeposits, biofilms or pathogens any of (a) in or on a living body including in or on a medical device or implement placed, inserted or insertable in the body, (b) in, on or in contact with an ex vivo bodily tissue or fluid, or (c) in, on or in contact with a medium or matter to be consumed, ingested by or exposed to a living being or entity. Also disclosed are medical devices, implements, and prostheses that can be kept in a safe defouled, pathogen-free or pathogen-reduced condition with the help of an electric field(s). Also disclosed are medical or industrial devices, implements, equipment-articles and storage/handling-entities that can be kept in a safely defouled, pathogen-free or pathogen-reduced condition with the help of a membrane or orifice that has one or more of the antifouling or antipathogenic attributes.

### BACKGROUND

The invention is mainly applicable to: (a) any medical device or material that is implanted in or juxtaposed to a human or animal tissue or body fluid, in vivo or ex vivo, permanently or for a short period, and (b) any useful article, material or device, medical or not, which is to maintain a human or animal health-safe unfouled state of cleanliness over a period of time. In these medical and non-medical applications it is known that undesirable fouling phenomenon can take place which might harm a patient, user or consumer of the compromised device, equipment or material processed by, transported or stored in contamination prone articles, equipment or environments. Such fouling phenomena include biofilm development and bacterial colony development. Such fouling can cause irritation and swelling, allergic reactions, toxic reactions, poisoning or infections. Other fouling phenomonena include the growth of deposits, crystals, or precipitates. These latter phenomena can physically plug devices and equipment or otherwise degrade its functioning, render it inoperable or encourage it to host bacterial colonies and/or biofilms. The hosted bacteria can then be passed into consumable products being processed by such equipment. In this manner if the fouled device or material is being used to process, store or transport food, water or breathable air, for example, the food, water or air may become unsafe as it will likely be fouled for any subsequent human or animal consumption, ingestion or contact. So we have contamination or pathogens which might be passed to a patient, customer or user indirectly as well as that on medical devices which may actually be implanted, used invasively, or used noninvasively causing a direct problem of in-body fouling. Some such medical contamination issues develop over extended periods of time for already-implanted or worn medical devices.

Items of the type (a) may, for example, become fouled by, encapsulated by or trigger the local or remote deposition and/or growth of biodeposits, biofilms, yeasts, fungi, mold, bacteria, infectious prions, microbes, pathogens, infections, plugging, clots, fibrosis, scar-tissues, cancerous tissues, fat-buildups, allergic reactions or toxic materials. They may also cause irritation, swelling or be the target of systematic bodily rejection. Such items are typically minimally-invasive or invasive implements if not devices implanted for extended periods. Some such articles cause problems even non-invasively as by long term contact to exposed skin or accessible membranes. Such contact to body tissues and fluids is typical of surface-worn and noninvasive devices and medical implements.

Items of the broader type (b) may, for example, develop several of the same problems as those of type (a), particularly such as becoming a host for biodeposits, biofilms, microbes, pathogens or allergens or becoming toxically-contaminated by bacteria, pathogens, allergens, chemicals or heavy-metals. Note that group (b) applications typically do not involve body tissue or body-fluid implantation or juxtaposition however potentially unhealthy deposits or fouling may similarly be forming or growing as for group (a) applications. Group (b) foulable entities are frequently pipelines, storage containers and packaging containing a similarly cofoulable medium or consumer product which a downstream customer later uses or ingests, such as water or milk. These types of passed-on or subsequent contacts present serious indirect risk. Type (b) items may or may not be medically related.

All of the fouling phenomena of groups (a) and (b) commonly involve a problem developing when a first material is perturbed by, exposed to, reacts with, is enclosed by or is held by a second material or device causing a reaction or response with undesirable or potentially risky consequences.

Relative to the applications of (a) a wide variety of medical catheters, drainage tubes, pumps, drug-depots, intrathecal catheters, cannulas, ports, other liquid or gas-flowing lumen devices, electrodes, stents, grafts and electrical/electrochemical/optical and other sensors of various types are used on and in human and animal patients or tissues/fluids in vivo or ex vivo. Such sensors include blood/tissue glucose monitors, electrolyte sensors, blood constituent sensors, temperature sensors, pH sensors, optical sensors, electrodes (receiving and/or transmitting), amperometric sensors, pressure and flow sensors, biosensors, sensing transponders, strain sensors, auditory sensors, activity sensors and magnetoresistive sensors. Fouling, such as biofilms, infection and fibrotic encapsulation, can degrade or deactivate such sensors, or perhaps worse, cause them to give false readings when partially blocked or fouled. Despite several decades of medical device and clinical progress, the infection, clogging, irritation, degraded-operation vs. time and/or rejection rates for most of these devices is still grossly unacceptable, and such infections and clogging are very costly and can even be life-threatening. Infection rates for some catheter devices are higher than 50% despite many decades of experience and hospital-based use.

Relative to the applications of (b), the reader will certainly appreciate the challenges faced in the safe operation of complex processing plants and containments for making, processing and storing materials such as food, water, meat, drugs and beverages. Also, the difficulty of keeping publicly-utilized facilities such as toilets, showers, water-fountains, air-conditioners and ducting and pools contamination-free is clear. The same applies to shared objects such as eye-protection and safety-wear. Recent experience also shows that even growing foodstuffs such as spinach can be contaminated as by wandering animals. Most of these applications require something more than a simple wipe with a germicidal. Many of them are simply not amenable to such measures, even if they were fully effective.

The present invention herein addresses these issues utilizing at least one of (a) An applied or induced electric field or (b) A Laplacian flowable orifice or membrane. One or both of these primary approaches or mechanisms may optionally and additionally be coupled with or used in combination with one or more of seven other preferred complimentary antipathogenic, pathogen-avoidance or defouling mechanisms. More new mechanisms are expected in the future which can also be used with the present invention. The applied or induced electric field is typically pulsed and the pulse lengths are chosen to selectively cause internal and/or external cell damage such as to pathogens or cellular deposits. The following reference teaches the biological mechanisms of the pulsed electrical field approach-particularly when extended to nanosecond regime pulsing which selectively damages and manipulates the cells inner workings or intracellular membranes and entities:
“Nanosecond, high-intensity pulsed electric fields induce apoptosis in human cells”, Stephen J. Beebe et al, The FASEB Journal express article 10.1096/fj.02-0859fje-published online Jun. 17, 2003.

The Laplacian orifice or membrane is typically a porous and permeable flowable (under the minimum Laplacian activation pressure) orifice or membrane, the orifice or membrane requiring an applied differential pressurization across it to allow for flowable medium ingress and passage. The Laplacian action, i.e. pressure-forced ingress of the flowable medium, will be used to at least provide our inventive antipathogenic or antifouling capability but may also be used for other device purposes such as serving as a pressure activated valve used for a non-cleaning purpose. Thus, the Laplacian action may be only part of our antifouling mechanism or may also or in addition serve a second useful purpose. The Laplacian orifice or membrane will typically be replacing a prior-art open-flow orifice such as in a catheter or an exchange-membrane as in a dialysis system. Laplacian behavior has a key inventive advantage in that it allows for automatic dewetting or the orifice when not in use, thus, making it more difficult to foul or host pathogens in a “dry” permeable material. It thereby can also act as a valve. Laplacian threshold (ingressing) pressure may be applied in any manner as by conventional fluidic or pneumatic pressurization or by electrophoretic, electroosmotic or electrorheological induced pressurization. Note that typically our Laplacian orifice will also allow for a mass-transfer or flow above the Laplacian threshold such that that flow state can be used as for a drain or drug-delivery lumen. Our inventive Laplacian membranes will typically be exchange membranes or mechanical filters which normally only selectively transfer certain species but above our Laplacian pressure threshold they transfer additional types or quantities of species useful for defouling or antipathogenic purposes. The additional species might be the parent fluid containing the mentioned species and that fluid flush-cleans the membrane or loads it with drugs for example.

So with Laplacian orifices or membranes we have a pressure-wettable and pressure flowable component. We can think of it as a pressure activated flow-switch which prefers to remain dry. We inventively utilize the dry vs. wetted state to either or both of a) provide for a dry surface which is not easily fouled and/or b) have the dry to wet transition serve as a valve to allow a flow, say of a bodily fluid to be drained or of a drug which defouls a lumen.

Examples of seven existing preferred optional complimentary defouling, antipathogenic or fouling-avoidance mechanisms useable in combination with the above two core elements is as follows:
1) Use of applied acoustical energy, said intensity or energy level causing one or more of prevention or removal of fouling entities and/or damage to pathogenic entities. Such energy might be non-cavitating or cavitating. Such energy may or may not be chosen to cause thermal effects such as thermal necrosis or ablation of cells. Such energy may or may not be chosen to cause sonoporation. Such energy may be applied from inside or outside the living body or from within or from outside the foulable processing/storage apparatus.
2) Use of applied ultraviolet light or UV, whether produced internally or externally, delivered to the living body or processing/storage apparatus. Ultraviolet light is known to damage cellular apparatus and chemical bonds and is being utilized in water purification for example. Such light might be pulsed or continuous for longer periods.
3) Use of engineered surface properties on in-situ surfaces such as use of biofilm inhibiting surface topography (e.g., roughness or porosity), surface-tensions, surface charge-states/polarizations such as provided by surface conditioning or voltage/current application, surface-activation, surface polarization, surface-coating, surface-composition control or manipulation of the materials or surface's inherent electronegativity. These can apply to one or both of external or internal surfaces and to pore surfaces.
4) Use of in-situ static or pseudostatic pressurization or shock events (pressurizations and shocks produced locally or remotely) These are different than (1) in that they are static or of typically low-enough pulse frequency (compared to ultrasonic vibrations typically utilized for (1) for example) such that macroscopic liquid flow-inertia can be overcome and macroscopic flow-blockages freed-up.
5) Use of an in-situ antipathogenic drug, chemical or radiation exposure, including any gaseous, plasma, liquid, semisolid or solid-state ionic exposure such as exposure to silver or copper ions or ozone, or exposure to damaging or ionizing radiation such as ionizing or nuclear radiation or nuclear particle fluxes. Drugs, ions or chemicals, for example, could be locally provided, as on/in the foulable device, or could be systemically provided.
6) Use of in-situ heating or freezing, possibly including the driving of associated thermal phase-changes or cellular damage, such as to cause cell death and/or surface-scrubbing explosive microboiling (e.g., via local or remote electrical resistance, radiant light, radiation, RF, acoustics, microwave or chemical-reaction sources).
7) Use of in-situ foulable device and/or tissue/fluid electrical or ion currents wherein an electrical or ion current is caused or maintained for a useful defouling or antipathogenic period.

By “in-situ” we preferably mean implemented on/in the foulable device, equipment, tissue or material while it is in the field being used or it is in/on the body. This does not, however, limit the invention from, also being employed offline such as during or after the manufacture or storage of a foulable material before the customer ever sees it.

By “in combination” we mean two or more mechanisms utilized sequentially or simultaneously regardless of schedule or time-delay between implementing or applying such two or more mechanisms. For such a combination at least one of the mechanisms will be one of the two core mechanisms (pulsed electric fields Or Laplacian orifices). One or both core mechanisms may also be used alone.

Such “combined” mechanisms may have additive effects or, even more attractively, have cooperating synergistic if not co-amplifying effects. In cases wherein the effects are synergistic, it should be noted that there are at least two ways two mechanisms “1” and “2” can be synergistic including: a) 1 and 2 are simultaneously implemented, at least in part, and they synergistically favorably interact in real time and/or b) 1 and 2 are interleaved or sequenced, at least in part, and at least one of them is favorably affected by a remnant or historical effect(s) of the other having been implemented before it. Of course, three or more mechanisms might have even more such favorable direct (or indirect) interactions among even more mechanism-combinations. A synergistic effect can include enabling another mechanism as opposed to, or in addition to, accelerating or amplifying another mechanism. The other enabled mechanisms effect's then might be additive or also synergistic with those of the first mechanism or with a third mechanism. In the latter case, one mechanism is doubly synergistic (enabling and amplifying) of a second. Any one or more mechanisms may provide additive or synergistic effects for any one or more other mechanisms-including directly or indirectly through intermediate or other mechanisms. Two mechanisms which favorably interact might not even need to be applied the same day. Further, only one of them may be provided in an inventive device or material, with the other being inventively applied systemically throughout the body. On the other hand, both may be provided locally in or by the inventive device or material.

Returning to various fouling and plugging phenomena, other fouling issues such as bodily irritation, redness and lipohypertrophies can be caused by even temporary percutaneous devices such as tissue-embedded drug-pump cannula. These issues can also be favorably addressed by application of one or more of the above mechanisms. Swelling, or at least swelling avoidance, may also be addressable using one or more of the inventive mechanisms. Note that we also define such abnormal collections of cells and tissues as fouling as it can just as well interfere with a device's operation or a person's well-being.

Further, relative to the applications of (b), a wide variety of non-implanted medical and non-medical items or articles of use can become clogged, fouled or pathogenically, toxically or allergenically compromised and therefore become unsafe to use, consume, operate or be directly/indirectly exposed to such as by consuming or utilizing a product-material processed through or manufactured in them or as by wearing such product. These include medical surgical tools and instruments, medical non-surgical tools and instruments, patient-care equipment, food-industry piping and storage equipment, drug-making plant machinery and piping, food and drug packaging and storage equipment, eating utensils, dental equipment, faucets, showerheads, toilets, saunas and pools, air conditioners and associated ducting, fish tanks, aquatic components, refrigerators, milking systems in dairies, toothbrushes, restroom facilities, cosmetic care implements and equipment, computer terminals and kiosks, personal-care implements and equipment and water distribution/storage facilities. The same taught solution mechanism or mechanism combinations are applicable to the both the group (a) and group (b) applications.

What applications (a) and (b) have in common is the actual or potential development or hosting of unhealthy or interfering contamination, deposits, growths or pathogens over time when a first material or device is exposed to a stored, passing, contacting or surrounding second material, medium or device. Typically, the materials themselves will interact but sometimes, for example, just the shape of a material component can cause plugging-deposition problems in or at the foulable component.

By “unhealthy” we mean the contamination is itself bad and/or the contamination causes a device, material or tissue to become bad or have degraded operational capability. Note that in the applications of (b) such as the case of, for example, a fouled milk-processing pipeline, the pipeline and the milk which comes from it may both be fouled-whether separately or one because of the other. So the fouled milk can directly harm the consumer in this case, not the fouled piping (device) itself—the pipe which may have initiated the milk to foul. Reduction of such transferred harm or risk is within the inventive scope.

In both of the application groups (a) and (b) above, patients or the public could be sickened, irritated or even killed by such issues going unaddressed or undetected. The state of the art in 2007 should not be allowing for thousands of people being killed by such fouling and/or infections per year and contamination rates on 50 year-old medical technology such as urinary catheters to still be causing 50% infection rates in some hospitals.

Using the invention one may also or instead have two or more mechanisms acting relatively independently on two different aspects of the fouling and/or pathogenic problem. For example, a first mechanism might kill or prevent some pathogens that may grow in/on biofilms while a second mechanism kills, prevents or removes the biofilm itself. Conversely, a first mechanism might act on pathogens and a second mechanism also acts on pathogens-both to either kill or prevent or one to kill and one to prevent. In either of these scenarios it may be the case that two or more mechanisms act synergistically. This is the case if the presence of the first (or the effects of the first having been performed) enables, improves or extends the desired effects of the second or vice-versa. Many of the mechanisms taught herein can act both antipathogenically and in an antifouling manner although it is not an inventive requirement that both abilities be used in a given application. As an example, cavitating ultrasound can kill pathogens but it can also clean biofilms off-of or from surfaces and pore spaces. One might use ultrasound cavitation, for example, in a given case wherein only one of those tasks is performed-or in another case wherein both tasks are performed.

Another key challenge for indwelling medical lumens, catheters or sensors is fouling, physical clogging-of or other functional interference-with their ports, flow-channels, orifices, sensing apertures or windows. Such clogging and interference may or may not involve biofilms, pathogens or bacterial colonies growing from, in or in association with biofilms. Frequently such clogging can also, or instead, be caused by particulates, fibrils and crystallites found in or formed in urine, blood, insulin, drugs and cerebrospinal (CSF) fluids. Sometimes systemically-delivered therapeutic medications can even enhance or cause such depositions perhaps even of accumulated or excess medicament constituents. The lumen flow orifices, being the first significant flow constriction seen by inward or outward flowed liquids or media, frequently act as deposition or physical-capture sites for such debris or precipitation. Part of this is due to flow stagnation perturbation phenomenon at the orifices. Note that a fouled orifice can also act as a biofilm breeder or an outright bacterial multiplication site. Of course an orifice not clogged by such debris can also still grow a biofilm which invites infection just like the rest of the exposed surfaces of the implanted device may do.

When biomedical devices are implanted in the body, they are subject to a “foreign-body” response from the surrounding host tissues. The body recognizes the implanted device as foreign, provoking an inflammatory response followed by encapsulation of the implant with fibrous connective tissue (or glial tissue within the central nervous system). Scarring (i.e., fibrosis or gliosis) can also result from trauma to the anatomical structures and tissue following implantation or can occur if the device is manipulated causing irritation due to the daily activities of the patient. When scarring occurs around the implanted device, it may become clogged or may fail to function properly. An inflammatory foreign-body response can also result in tissue necrosis. These phenomena can also cause plugging or fouling. Thus, the invention can also address undesirable aspects of the body's natural responses, at times and at locations where these responses can cause fouling/plugging or pathogen hosting problems. Clearly, such swelling and scarring can act as a foulant and is also an object of the invention.

The present inventors note that by “defouling” we mean the elimination of harm caused by deposits or growths. There are some instances, for example, wherein an infected interface will have its pathogens killed by the invention and the pathogen-laced interface will be replaced with pathogen-free scar tissue or blood or interstitial fluid, for example. The point here is that the previously pathogenically fouled interface is not necessarily always “physically cleaned”; it is neutralized from causing the harm that would have been caused had there been no intervention. In this case, the pathogens are killed or reduced to non-harmful levels.

The above cited Beebe electric-pulsing paper mainly addresses the newly discovered scientific phenomenon wherein ever-shorter applied or induced electric-field pulses begin to damage or alter the inner workings of cells as opposed to damaging or altering their external membrane regions outright as does conventional electroporation which typically uses much-longer pulses or continuous currents. In essence, the Beebe paper explains that for very short pulses of 1-300 nsec at applied electrical fields of up to 300 KV/cm, cell external membranes become electrically transparent such that the cells inner workings experience the applied pulses. This is because the extremely short pulses cannot achieve a significant stable charge state on the outside cellular membrane in that short of a time. So unlike conventional electroporation for millisecond(s) and longer pulses the external membrane is not outrightly porated or ruptured but rather the cells inner contents, including possibly intracellular membranes, are damaged or altered. In fact, the work described in the Beebe paper discovered that apoptosis can be effectively induced by such nanosecond regime pulsing in most or all cells whereas electroporation typically kills only a portion of treated cells unless extreme currents are applied. Beebe does not teach the application of the nanosecond pulses to our applications herein and his is really a scientific study of first caliber. Inventors herein recognized that such nanosecond high-voltage gradient pulses can be applied to devices such as catheters, needles and bandages and that for many of these applications one may utilize the human body as one of the electrodes simplifying complexity. This is because the human body has enough momentary capacitance to allow enough charge-carrier flow to create such nanoseconds-lasting voltage gradients near and at our electrodes.

The “Laplacian pressure” is the ingress pressure necessary to wet an internally unwettable permeable flow conduit's interior surface(s) such that through-flow can then occur. In our inventive applications, the Laplacian orifice or membrane will typically comprise an aqueous-unwettable, poorly wettable or species-specific transporting polymeric material having a permeability via interconnected pore spaces. We recognized that if a Laplacian orifice or membrane is not wetted, it is much less likely to become fouled. Thus, a switchable orifice that flows only when necessary can reduce internal fouling and pathogenic activity. Further, such an orifice can easily be made of unwettable material such as porous Teflon®. Porous Teflon is known to inhibit water ingress and is utilized in grafts. We utilize Laplacian orifices to reduce orifice fouling as by the ability to dispel fluids therein and the ability of such permeable orifices to physically or mechanically filter particulates. Porous membranes are used widely to selectively remove or add (exchange) species from one medium to another such as by osmotic or electroosmotic action. Dialysis membranes and membranes used to desalinate sea water are two good examples of this. At any rate, such membranes frequently have a finite lifetime because they get fouled or contaminated-whether by pathogens or inert deposits of various sorts. Our Laplacian pressure threshold allows us to force-wet such membranes to clean them, load them with species such as antipathogenic drugs, or use them as valves for mass-transport of different species or larger quantities of the same species.

Biocompatible Materials Inhibiting Coatings and Surface Finishes:

Engineered “biocompatible” materials that are currently regarded as suitable for one or both of temporary or long-term implant-use in the human (or animal) body are well established. These include, for example, polyurethanes, polyethylenes, polyetheretherketones (PEEK), silicones, titanium metal, some ceramic materials used in artificial joints, and porous or “expanded” PTFE Teflon™ known as ePTFE as is used in many grafts. Others include FEP, PFA, PLA, PLGA and Dacron™. Many of these materials are biocompatible, meaning they have little or no toxic reactive effect on the body (nor the body on them) for a useful period. That does not mean that they do not ever develop biofilms, never cause any reaction, nor have any of their pore-spaces (if they are porous/permeable) plugged. In fact, many porous implanted materials are designed to have their pore spaces eventually plugged by invasive tissues, thus, establishing the implant as an integral and mechanically fused member of the body. Our invention herein, as it regards plugging, is aimed at preventing or delaying such plugging when plugging is not desired in a given timeframe, as in the case wherein the pore space, orifice or lumen is designed to allow a prolonged liquid-phase or gas flow. “Permeable” herein means pore-structures or internal paths allowing for a flow, transport or diffusion significantly larger than that through or in a comparable non-permeable structure or material. Most often, these permeation paths are tortuous and flow-connected allowing for some net mass flow, diffusion or flux from a first pore directly to neighboring second and third pore and so-on. “Porous” merely means having pores or included holes, which might or might not be openly locally flow-connected. A chunk of solid material having only a porous surface region or layer is not permeable through its overall bulk thickness; however, it might be permeable within its porous surface regions, both laterally and thickness-wise if the near-surface and surface pores are connected in both of those directions (i.e. the porous region is also permeable). Thus, our flows, diffusions or fluxes will at least flow or diffuse within such locally open permeable regions if not through entire bodies made to be permeable throughout. We include in the scope of “permeable” a material or article which is arranged to have microminiature, microscopic or nanosized pores or flowpaths that are artificially or naturally made or grown, including those whose one or more pores are not openly locally connected within the material bulk and each are thus, independent of each other. For such a material to still be permeable, some of the separating walls or isolation membranes defining the pores themselves need to be diffusive to the moving species as by osmosis or diffusion. So in this special case, it is (at least) the permeable-material walls or membranes between and defining the pores that allow a flux or diffusion through them. Note that this can take place even if the pore spaces are indeed all openly pore-isolated from each other so there is minimal or no direct pore to pore flow between adjacent pores—only interpore leakage or diffusion across the interpore isolation walls or membranes. So permeable means that a flow, mass transfer or diffusion is at least taking place one or more of: a) through flow-interconnected pores or pore channels b) through pore or interpore membranes or walls, c) through pore membranes and the pore paths or channels themselves (i.e. (a) and (b)). We discuss below Laplacian fluid ingress pressure and it should be noted that a Laplacian pressure can exist for a permeable unwettable (or poorly wettable) material whether or not any mass-transfer or diffusion takes place across the interpore membranes or isolation walls. In other words, Laplacian driving fluid-ingress pressure can cause an inward saturation of a permeable material but may or may not be enough to further drive flow out the other side.

Many implantable materials, particularly the useful flexible polymers, plastics, rubbers and elastomers used for most types of catheters and drainage tubes, are easily fouled by biofilms which naturally agglomerate and grow upon and/or in (for permeable polymers) these materials when implanted in the bodily or body tissue/fluid environment. Such biofilms are superb hosts for infection as they actually protect bacteria from attack relative to the case of if the bacteria were instead free-floating planktonic bacteria. This protection factor between free-floating planktonic and biofilm-based bacteria can easily be a factor of 2-50×. Thus, once an infection gets started in a biofilm, it is conventionally (in the prior-art) extremely difficult to kill it without using dangerously high doses of antibiotics or other high doses of antipathogenic agents, radiation-energy or heat.

It has been known that some materials naturally inhibit or slow (but typically do not completely indefinitely stop) biofilm development-most likely by inhibiting the initial biofilm cellular agglomeration stage. One such material is Teflon™ and it is thought that this effect is associated with Teflon's very poor wettability by aqueous liquids. Other fluoropolymers, for example, exhibit similar behavior to various degrees in approximate accordance with their degree of aqueous nonwettability. Such lubricious or poorly wettable materials include PTFE, FEP and PFA for example. Poorly wettable materials typically inhibit biofilms to various degrees. Poorly wettable materials used in the invention, for example, may be bulk fluoropolymers, bulk non-fluoropolymers or similar materials deposited or coated on component surfaces as by solvent-based dipping processes, vapor deposition processes or plasma-assisted deposition or surface-conversion processes. Coated surfaces might include exposed or external surfaces or may instead/also be porous or permeable internal surfaces for example.

Furthermore, it has been known that certain regimes of surface roughness or bulk-porosity on or in any given material can also topographically inhibit the initial agglomeration stage of biofilms. Some polymers, metals, glasses and ceramics of controlled microroughness or porosity have demonstrated this “topographical” biofilm suppression mechanism. This topographical mechanism is thought to introduce physical interference to the ordered spatial agglomeration of the otherwise initial continuous biofilm. It is understood for this mechanism that the desirable topographical feature size (e.g., mean roughness) is ideally on the approximate order of the mean size of the depositing cellular, platelet, fibrous or cluster-like biofilm entities to be inhibited from depositing. This seems to offer the most interference with an otherwise orderly deposition nucleation process. Such mean applied roughnesses are typically measured in the micron range. We note that the use of a permeable material whose pore size is small enough that a particular pathogenic and/or fouling entity cannot enter or grow is also within the scope. Such growth limitation could be because the mean pore size is much smaller than the mean cell or globular size of the pathogen or biofilm constituent for example. So this is “topographical” interference in three dimensions with even a single cell-let alone an agglomeration thereof. Note that such a material would both prohibit the inflow of cells of that size and would prevent the growth of cells therein to that size from an initially smaller size. This growth restriction may usefully kill the cells or otherwise render them harmless.

Despite these beneficial low-wettability and controlled-microroughness tendencies toward biofilm and infection delay or inhibition, these tendencies are not by themselves typically sufficient for longer-term implants to remain totally biofilm, pathogen, debris and infection free. By “longer-term” we typically mean a few to many days, weeks, months or years. Unfortunately, some medical devices, such as chemical/electrode glucose sensors, can be measurably diffusively and electrically interfered with by even small amounts or monolayers of biofilm in just two or three days of percutaneous insertion. Such interference throws off the calibration of the sensor or forces it out of its normal range of operational parameters wherein an acceptable signal to noise ratio and/or accuracy can be attained.

Thus, recently, there has been a surge in work aimed at enhancing the biofilm and microbial resistance of indwelling medical lumen-based devices and even non-lumen devices. Often this work has involved adding antimicrobial or hydrophobic/hydrophilic coatings to the exterior and/or interior surfaces of indwelling lumen materials if not to the bulk of such materials. Often such antimicrobial or germicidal features are implemented in, inside-of, or into the surfaces of nonporous, porous and/or permeable materials such as ePTFE, PTFE, FEP and PFA.

Drug Based Chemically/Ionically Active or Radiative Inhibitors:

Known methods of preventing the production of excessive fibrous or scar tissue foulant on biomedical devices and implants involves coating them or surrounding them with agents like cytostatic antiproliferative drugs such as sirolimus, analogs of sirolimus, tacrolimus, antiplatelets, antihistamines, analgesics, anesthetics, angiogenesis inhibitors, chemokine receptor antagonists CCR, immunomodulators, cyclin dependent protein kinase inhibitors, heat shock protein 90 antagonists, cell cycle inhibitors, antibiotics, antivirals, estrogen receptor agents or antagonists, antifungals, 5-lipoxygenase inhibitors and antagonists, corticosteroids, Bisphosphonates, HMGCOA reductase inhibitors, phosphodiesterase inhibitors, inosine monophosphate dehydrogenase inhibitors, NO antagonists, Factor Xa inhibitors, famesyltransferase inhibitors, leukotriene inhibitors, NF kappa B inhibitors,EGF (Epidermal Growth Factor) receptor kinase inhibitors, elastase inhibitors, thromboxane A2 antagonists, TGF Beta Inhibitors, Fibroblast Growth Factor inhibitors, Tyrosine Kinase inhibitors, TNF alpha antagonists and TACE inhibitors, p38 MAP kinase inhibitors antithrombotics or anticoagulants. Fibrosis (replacement of injured cells by connective tissue) involves inflammation, migration and proliferation of connective tissue cells such as fibroblasts or smooth muscle cells, the formation of new blood vessels (angiogenesis), deposition of extracellular matrix (ECM), and remodeling (maturation and organization of the fibrous tissue).

Further, silver and silver-copper solutions are utilized in several antibacterial or germicidal preparations. Silver and silver-based ions and radicals are known to have a strong antibacterial behavior.

Ultraviolet, nuclear and particle radiation are also known to have useful pathogenic effects with ultraviolet light being widely utilized in water purification systems. Because of nuclear and particle methods being more expensive they are less commonly utilized but still have known and predictable pathogenic activities. In the production of medical devices Gamma radiation is often utilized to sterilize entire pallets of packaged devices.

By “chemicals” we mean the at least temporary introduction (or in situ production) of any chemical, compound, ion or radical, natural or engineered, stable, metastable or unstable that damages or attacks pathogens or deposits potentially or actually hosting them. Such chemicals may be delivered alone or in solution for example. They may also be delivered by known time-release methods. They may be in any physical form including liquid, gas, gel or ionics. They may also be in the form of prodrugs or prechemicals which are not active until they are needed.

Ultrasonic Inhibition, Destruction and Alteration:

Ultrasound has been known to enhance many useful processes such as diffusion, dissolution, erosion, cell-membrane penetration, osmosis, liquefaction, transfection, cell membrane modifications and some immune mechanisms.

The historical challenge to the use of ultrasound for the general applications herein has been to get the required ultrasonic or acoustic intensity where it is needed without causing excessive heating of polymeric catheters and untargeted tissues. Other challenges involve transducer miniaturization, the challenge of battery-supported operation and patient protection against high voltages. There have been several unproductized inventive attempts at ultrasonically cleanable or antifouling catheters (see, for example, below).

It should be obvious to anyone with an understanding of physics or mechanics that a long rubbery catheter is not going to conduct ultrasound intended to defoul very far along its length without huge attenuative losses. Reasonable countermeasures to reduce such losses by inserting low-loss acoustic waveguide wires in the catheter work better but still have problems with bends in the catheter whereat most of the energy is lost, catheter heating (and subsequent tissue heating), transducer-size and cost. There has never been an in-situ ultrasonically cleaned lumen device productized and sold successfully for these and other related reasons-despite numerous issued patents. What is probably required is the use of very low-loss novel catheters and/or placement of transducers on the catheter itself. These may still not be sufficient and will be expensive and complex and not as flexible as today's polymeric catheters. It is unlikely that a catheter outer diameter can be acoustically excited enough to prevent surface deposits and/or kill bacteria without irritating or injuring the patients tissue at that site-unless one utilizes very high frequency having minimal tissue penetration which would certainly require in situ ultrasound generation. By “very high frequency” we mean preferably tens of megahertz if not hundreds of megahertz which would be useful for very shallow, i.e. biofilm heating. Cavitation will require much lower frequencies as is known but has the danger of damaging or irritating healthy tissues at a distance and in the nearfield. We note that when used in combination with our two primary inventive approaches (pulsed electric fields and/or Laplacian orifices and membranes), that the required acoustic power to gain a particular level of pathogen and fouling avoidance/safety can be reduced from using acoustics alone. We cite some unproductized, ultrasonics-only patent references immediately below:

Greenfeld et al related U.S. Pat. Nos. 4,698,058, 4,906,238 and 5,061,255:

These patents teach the use of acoustic waveguide delivered vibration to the clogging orifices of catheters. The waveguide is either a catheter-embedded wire or a catheter-embedded liquid-filled conduit. The major issue with these improved designs (relative to those with no waveguides) is still that little of the ultrasound will make it to the tip of an extended rubbery catheter in-phase and much of it will be attenuated as heat before it progresses that far, especially at bends. This is particularly the case using Greenfeld's recommended shear waves which are very efficiently turned to heat and efficiently coupled to surrounding materials. Further, Greenfeld intersperse a body of rubber catheter material between the end of their wire/fluid lumen and their target orifice. These design decisions will absorb a lot of ultrasound in particular-turning to heat adjacent the orifice. In the case of the embedded wire this is because acoustical energy will leak laterally into the rubber catheter body along the entire length of Greenfeld's acoustic wire. In the case of the liquid filled lumen the compressional waves will be easily converted to sideways waves or shear waves. Further, Greenfeld's frequency-scanning will not be able to excite sustained cavitation if that is desired. Only very low frequency waves might be delivered and those will have wavelengths bigger than the size of the features to be cleaned such as bacterial cells and biofilm thicknesses. Thus, the physically-large waveforms will merely vibrationally translate the orifice/deposits not very effectively cleaning them and unacceptably vibrating and irritating patient tissues adjacent the vibrated catheter. Further, Greenfeld does not teach how to make an acoustically matched transducer nor a miniaturized transducer nor one that might be disposable. Further, the suggestion to locate the acoustic exciter well outside the body, if not in another room, is totally unworkable in view of realistic acoustic waveguide loss mechanisms. The acoustic feedback schemes described therein are unworkable in that an uncontrolled and variable amount of acoustic energy will also be lost along the return fiber and because little of the cleaning energy will even be coupled into such a feedback fiber. The optical feedback detection method is more technically realistic to achieve feedback yet still complex if it requires interferometry. U.S. Pat. No. 4,698,058 is primarily directed at orifice cleaning whereas U.S. Pat. No. 4,906,238 is primarily directed at cleaning of the external diameter or surfaces of catheters by utilizing the same vibratory sources and a series of surface grooves. It isn't obvious that the groove system will not actually encourage even more bacterial growth. U.S. Pat. No. 5,061,255 is similar to U.S. Pat. No. 4,906,238 in content.

Zumeris et al, Nanovibronix Inc, US Patents Pending US2005/0095351A1, US2005/0268921A1 and International Applications WO 03/099100A2, WO 2005/117156A2 and WO 2006/053004A2:

These patents are directed to utilizing ultrasonics to prevent bodily entry of bacteria or to disperse bacterial clumps. The actual case is that some of the bacteria growing on catheters come from inside the body and that breaking up bacterial colonies will also likely result in smaller (initially) infections. Significant portions of bacteria come from the practitioner's hands or gloves and get inserted along with the catheter.

In any event, the above conclusions for the previous patent references also apply here.

Ultraviolet Light or UV:

Intense UV in the 255-280 nm wavelength regime is known to be able to kill many bacterial species such as those in water, wetted or aqueous environments. Thus, in the construction of pure water systems, intense UV lamps are employed to directly kill waterborne or otherwise wetted bacteria and possibly create some immersed ozone. Obviously, the fouled medium through which the UV is to be passed must be relatively transparent to UV wavelengths. This combination of transparency requirement and the required high applied intensity limits the applications to which UV can be applied. A severe limit is also presented because in most situations one desires to kill only bacteria among healthy cells, without killing or seriously damaging the healthy cells. In the water purification case this is not a problem. Short of killing cells, one could also damage healthy cells unintentionally. Within our inventive scope herein is the use of wavelength-tuned UV to kill or disable specific pathogens having particular wavelength susceptibilities as well as the use of broadband UV. Such UV would be used in combination with one of the two core inventive elements of electric field pulses and Laplacian orifices.

### BRIEF DESCRIPTION OF THE DRAWINGS

FIG. 1 depicts a catheter having the inventive pulsed electric-field defouling mechanism at an orifice as well as a biofilm resistant surface property on an external surface portion.

FIG. 2 depicts a catheter having the inventive Laplacian orifice as well as a biofilm resistant surface property and an impregnated antipathogenic species on a surface portion.

FIG. 3 depicts a needle-style cannula for drug delivery having by itself the inventive defouling electric-field mechanism at the drug emanating tip.

FIG. 4 depicts a glucose sensor for diabetics having both a pulsed electrical cleaning or antipathogenic capability as well as an ultrasonic biofilm disruption capability drivable from outside the body.

### DETAILED DESCRIPTION

We utilize at least one primary inventive mechanism of an electrical field, preferably pulsed, and/or a Laplacian orifice or membrane to avoid, inhibit or kill fouling deposits or pathogens.

In the case of the pulsed electrical field, we preferably utilize a pulse length or frequency having a known relationship to a cell membrane charging time such that we can assure one or more of: (i) cell-interior damage only, (ii) cell-interior and cell exterior membrane damage, or (iii) cell exterior membrane damage only. Most of our preferred electrical pulsing applications will utilize at least some cell-interior damaging pulses as they have a higher kill-rate for cells. These are known to result in apoptosis or efficient cell death. We anticipate two preferred general types of electric antifouling and/or antipathogenic treatments.

Electrical Approach 1: Two or more man-made electrically conducting members or wires are routed along, in or through a device or article which is to receive preferably-pulsed electrical cleaning or defouling. Essentially, we apply or induce an electrical field between or across the two wires or electrode members-preferably at locations whereat the wires are bare or without insulation. In this manner one could have an entire wire pair be bare for a full cleanable or defoulable lumen-length or could, for example, have just a localized bare region of the wire pair be bare of insulation whereat localized cleaning or defouling is to take place, such as at an orifice. There could be more than two wires such that the electric field polarities can be varied across, through or around a device between two or more pairs or groups for example. Electric fields created across or by the electrodes serve to defoul the device and/or surrounding tissue/fluid.

Electrical Approach 2: Electrical fields, preferably pulsed, are applied or induced as in Approach 1 however the field(s) are applied or induced instead using at least one electrode comprising a conductive medium or material such as saline or urine in an insulated lumen. Thus, two sub-embodiments of this are
a) A liquid saline electrode comprising liquid in an insulated catheter lumen delivers an applied potential to a catheter orifice having communication with the body tissue or fluid. The saline emanating from or resident in the orifice allows an electric field to be set up in the tissue/fluid at/outside the orifice via capacitive charging. Thus, capacitance of tissue/fluid acts as a virtual second electrode. Assuming the orifice is localized it acts as a point source of potential and capacitive-charging momentary current. Thus, this field and current drop off roughly as 1/r³ moving away from the orifice into the tissue. Note that depending on the lumen diameter vs. the orifice diameter and therefore the voltage gradient provided within each one might damage or kill pathogens or cellular matter both in/at/outside of the orifice as well as inside the lumen.
b) A man-made electrode delivers a potential to a location whereat it is allowed to electrically leak into tissue/fluid or device portions which are to be defouled. For example an electrode wire could be routed inside and down a rubber catheter wall to the catheter tip or orifice. At the tissue-exposed tip or orifice, the potential again causes a 1/r³ field to be set up wherein the tissue/body fluid acts as a capacitor, allowing for a momentary current flow.

In any of these electrical approaches, the applied or induced field may be applied from a point, from a line, from an area, or from a three-dimensional (3-D) shape. As used herein, “from” means the driving electrode, in the orifice case, produces a 1/r³ field outside the orifice in whose potential and momentary current scale with 1/r³ roughly. In the case of the application electrode being an elongated wire or elongated conductive cylinder (such as a metal needle), one would then have roughly cylindrical 1/r² electric fields and momentary currents. As we stated, the delivery electrode might be metallic or ionically conducting, for example. The electric field shapes for different shaped electrodes are well known.

Before proceeding further, we emphasize that these or other electrodes may possibly also or instead apply sustained or CW DC or AC potentials and/or currents. For example, a DC voltage will induce a DC electroporating voltage and current which may be useful for electroporation or phoretic driving of drugs or bodily species inwards or outwards. A high electric current applied to a fouled device surface could cause irreversible permeabilization or electroporation of the microbial cell membranes and even directly oxidize cellular constituents. This would render the microbes contained within the force field of the biofilm more susceptible to oxidative and osmotic stresses, and potentiate the effect of treatment agents such as antibiotics by facilitating their diffusion and entry into the cells It is also known that surface charges themselves can inhibit bacterial survival and/or attachment, even without current. Because most microbes specifically bacteria carry a net negative surface charge, negatively charged surfaces discourage bacterial adhesion by favoring repulsion, while positively charged surfaces promote it. An initial negative charge therefore may delay the formation of biofilm by preventing microbes and mineral deposits from forming or adhering onto the surface of a membrane. In this way, we expect that biofouling would be greatly reduced.

Colonization, however, is preferred generally on negatively charged surfaces. Positively charged surfaces have been proposed to impede the colonization of negatively charged microbes by exerting strong electrostatic attractive forces on them and thus preventing the elongation and division needed for growth. Thus, we propose in certain circumstances that in the first few hours to days of implantation of a material, a net negative charge might be beneficial to delay formation of the biofilm. However, once biofilm formation has occurred, a net positive surface charge might inhibit colonization. Such DC applications can be combined with our short nanosecond regime pulsing.

In addition, a particular microbial strain isolated from the surface might dictate the surface charge. For example, Jucker et al. have tested adhesion of the Stenotorphomonas maltophilia to glass and Teflon. This bacterial species was isolated from a catheter specimen from a patient with a suspected urinary tract infection. Indeed, Stenotorphomonas genus is known to be involved in human infections. The overall positive surface charge of this organism at physiological pH can promote primary adhesion to negatively charged materials. Therefore, if infection with Stenotorphomonas maltophilia had occurred a negative charge might be used to inhibit further colonization or if it was likely to occur the surface would be initially made positive to impede adhesion through repulsion. Included in our inventive scope is the use of photosensitive materials which become charged or conducting with the application of light. Such materials or coatings thereof could act as electrodes or conductors.

A drug, medicament or fluid passed into or through an inventive device may be ionic in nature in that in the device and/or body it takes an ionized form. If we have the means to charge the surface of our device, then we can attract and hold such species acting as a depot or we can repel them and drive them into the surrounding tissues.

By “pulses”, regardless of their length, we include positive polarized pulses, negatively polarized pulses, alternating polarity or bipolar pulses, alternating but separate positive and negative pulses and repetitive pulses of any type. Repetitive pulses could include a repeated application of a given set of pulses such as one positive and one following negative pulse. As mentioned earlier, pulses may be accompanied by a DC or offset voltage or a net charging effect due to asynmmetric pulsing voltages.

Returning now to pulsing application, as with switching power supplies, the high voltage needed to deliver the pulse(es) could be developed by first establishing a large current in an inductor and then switching one end of the inductor to a conductive fluid. This arrangement is also similar to an auto ignition which actually contains two windings in order to achieve a higher step-up ratio.

If the conductive or semiconductive fluid is not conductive enough, then a man-made wire could be run part way down the catheter and then the fluid could carry the pulse the rest of the way. Many IV fluids that are ion-balanced with the patient's body such as saline have appreciable conductivity useful for this application. Many body fluids also have good conductivity such as urine. Within our scope, of course, is the rendering of drugs, medicaments, nutrients or other flowable media conductive as by introduction of ions or physical conductors therein. In this manner, a drug delivery catheter could clean its own tip. Even some gases such as CO₂ will form conductive ions in aqueous environments.

The electrical pulse length is a main factor in determining what type of alteration or damage is done to cells and/or their surroundings. Per the Beebe paper, nanoseconds range pulses cause damage to cell innards whereas longer milliseconds or longer pulses cause outer cell membrane wall damage or alteration. Short pulses, as mentioned, may, for example, be applied using only a single man-made electrode and a second capacitive virtual electrode or by two or more provided man-made electrodes. Again, electrodes may be, for example, metallic or ionically conductive.

The invention herein may apply or induce electric fields which any of a) cause cell internal damage or alteration, b) cause cell membrane exterior damage or alteration, or c) both (a) and (b).

Longer pulses such as for external cell damage or electroporation of the outer cell membrane wall will typically involve electrical currents which are passed between two provided or available electrodes. The user may apply simultaneous or sequential combinations, if desired, of such short and long pulses. By “pulse” we mean a waveform. Thus, a pulse could be anything from a lone sine wave or square wave a nanosecond long to a CW or continuous wave string of such pulses lasting milliseconds or longer. The induced or applied electrical fields may be unipolar or bipolar, and might have AC and even DC components. We note that electrophoretic and iontophoretic phenomenon can be driven by such sustained fields having at least a time-averaged polarity bias. Cell rupture and transfection has also been shown to be caused by such applied or induced fields. Net DC biases can also be used to drive drugs or beneficial ions such as silver into tissues or lumens.

So the tissue or cell-altering electrical mechanisms may include interior-cell alterations or damage and exterior-cell alterations or damage as well as the through-cell and/or intracellular manipulation or transport of ionized or charged species such as of an iontophoretic drug utilizing an applied electrical field. We expect that these electric fields will be utilized to not only damage or destroy pathogens and undesirable deposits or depositables but to also deliver drugs and perform therapies.

So it is important to note that the impetus for the invention is in fouling avoidance and reduction but that many such applications are indeed in drug delivery, cell-therapy and medical device applications addressing therapeutic or surgical purposes. Thus, the electrical constituents of our antifouling invention may also, simultaneously or sequentially, be utilized for those therapeutic or surgical purposes. One excellent example would be a drug delivery catheter which is kept defouled by the invention but which also utilizes iontophoretic mechanisms to aid delivery or activation of a drug. The same electrodes(s) may be utilized, in part or in whole, for both.

Electrical pulses, fields or electrode-conductivities might even be generated utilizing photoconductive materials. In that manner one can do so in a non-contact manner, even through the skin for some distance depending on excitation wavelength. Near infrared wavelengths are particularly good for such penetration. The use of any type of ambient, therapeutic or surgical-light to provide such charging or potential is within our inventive scope even if the light has another unrelated purpose.

In the case of utilizing a Laplacian orifice or membrane we utilize a forced-wetting phenomenon to switch a flowable medium or species therein such as a liquid into and/or through the orifice. The forced wetting or permeable-material intrusion can be done as by using applied fluidic or pneumatic pressure, applied vacuum or using electroosmotic, electrophoretic or other electrorheological effects. We prefer conventional pressure gradients or electrowetting mechanisms. In this manner, we have a switchable orifice or membrane which can be maintained in a medium-free (e.g., liquid-free) state when not needed. We note that our orifice or membrane will typically be fabricated of a permeable material and have poorly wettable interior flow passages thereby presenting the Laplacian pressure head necessary for fluid or species ingress above the Laplacian threshold pressure. We expressly note that the invention may utilize a non-Laplacian orifice or membrane such as a wettable permeable polymeric, ceramic, metallic or glass orifice. Any permeable orifice or membrane used in the invention, Laplacian or not, might have an electrical field applied across it (a primary inventive mechanism) to damage, disrupt of kill resident or transiting pathogens, growths or deposits. Note that a permeable orifice, Laplacian or not, with an electrical field applied across a portion of it assures maximal damaging exposure of passing and resident pathogens.

### Other Terminology Used Herein

### 1) Bodily Tissue/Fluid Perturbing Material, Article or Device

Any material, article or device in any amount, concentration, shape, form or size placed, transported to, or becoming situated in or in contact with any body or body-matter, in vivo or in vitro, which causes any response by the body or body matter that would not have occurred without that material's, article's or device's placement or contact or biologically or physiologically-communicating juxtaposition. Many if not most of such responses are foreign body responses wherein the body has recognized the object or material as foreign and is trying to protect itself from it. Additional responses, not necessarily involving the body's foreign object response or immunological response, could, for example, include corrosive phenomena, oxidation phenomena and other phenomena which are a result of the unavoidable and possibly non-immunologically driven diffusion of constituents or species from the body into/upon the exposed device/material and/or vice versa. Note that these transports or flows of fouling species do not necessarily require the typical immunological foreign body response to occur. The same can be said for the precipitation of excess patient calcium in an orifice or lumen. The result, however, is no different—an undesirable or risky change has taken place due to the perturbing event. Placement or contact of the perturbing material, article or device may be as by implantation, ingestion, inhalation, aerosol transfer, air-transfer, injection, physical juxtaposition or contact to the skin, percutaneous puncture, intraluminal delivery, blood or mucous contact, tissue absorption, or delivery by natural bodily mechanisms such as blood-borne transport. Such placement or contact may be purposeful or accidental.

A perturbing material, article or device is typically a part of a useful device, component or tool or is a useful material that is at least temporarily, intentionally or unintentionally, placed or situated, at least partly, in or in contact/communication with a human or animal body, body tissue, body fluid or with a constituent thereof. Examples include sutures, pacemakers, vascular catheters, spent bullets or shrapnel still in the wounds, percutaneous glucose sensors, drug-pump related delivery cannula, bandages, contact lenses, clothing, jewelry, hearing-aids and drainage catheters. Included is any material coming in contact with bodily tissue or fluid anywhere—even in vitro as for a dialysis machine, a urine drain-bag or a lab-on-a-chip for testing an ex vivo sampled bodily fluid specimen. Aerosol, air or physical-contact pathogens could include radioactive materials, particulates (e.g., diesel soot), man-made nanoparticles, bacteria or viruses from other persons. Typically, the bodily tissues and fluids discussed in the invention are living or viable but we include any current or future nonliving, nonviable or out-of-body stored or cultivated versions or constituents thereof having useful health, scientific or industrial benefit to someone and subject to taking part in or being involved with undesired fouling, plugging deposition, infection or growth occurrences risk. The response(s) to such perturbations typically involve one or more of biodeposits, infection, rejection, toxicity, plugging, clotting, fibrosis, fat-buildup, allergic reaction, scarring, necrosis, swelling, or irritation at or initiated from the perturbing material's location. Perturbing materials also include engineered (artificial) and human/animal sourced implanted and transplanted organs and tissues such as ePTFE and Dacron™ grafts and metal stents such as those placed in diseased lumens.

Note that skin-surface mounted materials such as bandages, drug delivery patches or pumps and wound closures are potentially perturbing materials, products or devices. Note also that the undesirable biodeposits, infection, rejection, plugging, clotting, fibrosis, yeasts, fungi, mold, fat-buildup, allergic reaction, scarring, necrosis, swelling or irritation response(s) will typically occur on/in or near to the perturbing material itself; however, in a few cases may occur remotely as triggered or enabled by the presence of or exposure to distal perturbing material(s). Allergic reactions even remote from the actual perturbing material are clearly a form of fouling as defined by the invention. These perturbations all harm or potentially harm the bodily tissues and/or fluids, directly or indirectly, immediately or over time. Some toxics concentrate in certain organs such as the kidney or liver and these are also in the scope.

It is one or both of a perturbing material, article or device itself (e.g., material composition, device material makeup, toxicity, ease of biofilm attachment) and/or the physical shape/form of perturbing material, article or device that may cause the harm or risk. Note that a plugging perturbing article might even be made of a highly biocompatible material which isn't itself an issue-the plugging being related primarily to the shape of the article in that case.

As a final example, a bandage which develops a yeast infection has, as the perturber, the bandage which has perturbed the normal exposed tissue surface and/or wound-inviting the pathogenic development of the yeast infection under and/or within the bandage.

### 2) Biofilm

### 2a) Bodily Tissue/Fluid Biofilm:

Any layer or film-like coating of a continuous or semicontinuous nature which grows, develops, precipitates or deposits in response to a perturbing material, article or device (see above definition). Such films usually grow, develop or deposit upon, in or adjacent the perturbing material, article or device itself but may, on occasion, grow or deposit remote from the perturbing material, article or device but still be biologically or chemically triggered or enabled by the presence of the perturbing material, article or device. Biofilms or bacterial clusters may also be shed from a perturbing device and colonize elsewhere. Commonly known biofilms include film-like living cellular coatings on long-term and even on some short-term medical implants. Note again that “perturbing” might simply physically comprise offering a preferred ready nucleation site which leads to plugging, stagnation or flow-constriction, i.e. it does not necessarily have to involve pathogens or toxicity. What is common to all bodily-formed films, whether biological or not in composition or species makeup, is that a bodily material or fluid, and/or transport path offered thereby, was required to cause it or enable it to form.

### 2b) Biofilm Not Requiring A Bodily Environment:

An example of this is a biofilm growing in a water faucet tap. A second example is a biofilm growing in a milking-line at a dairy. A third is a biofilm growing in an air-conditioner duct. These form despite the lack of a bodily environment. These films may host pathogens or be comprised of pathogens. Pathogens include viral and prion constituents and not just bacterial constituents, i.e. any harmful species. Legionnaire's disease from ductwork is a good example.

### 3) Biodeposit

### 3a) Bodily Tissue/Fluid Biodeposit:

Biodeposit is a broader term than biofilm in that it includes all shapes and sizes of growths, precipitates or deposits formed in response or reaction to perturbing materials, articles or devices. They do not have to be island-like, semicontinuous, film like or continuous in nature and may be inert, nonliving or nonviable discrete particles, discontinuous coatings, fibrils or tangles, or concretions for example. They might also be continuous. Biodeposits include infectious masses, attached or immobile thromboses, concretions, calcium masses, precipitated insulin crystals in an insulin delivery cannula, precipitated urine crystals in a Foley drainage catheter, fibrotic masses, protein-rich plaque/fibril/tangle masses such as seen in many neurodegenerative diseases, blood clots, kidney and gallstones and related concretions and corrosive products. Corrosion can possibly form as a film on a metallic implant component. And of course also included in the definition of biodeposit are the above widely known problematic and persistent cellular biofilms. The common theme of our definition, as for biofilms, is that a bodily fluid or material was required to cause or enable the biodeposit. Note that tumor cells shed from a metastatic parent tumor which deposit elsewhere away from the parent tumor and might multiply are clearly biodeposits by this definition. Fibrous connective tissue, lipohypertrophies, ECM or extracellular matrix and scar tissues are also forms of biodeposits per this definition. Note that biodeposits may be discretely situated in one precipitate, lump or film or may be distributed or diffuse in their location.

Ingested or breathed particulates are also biodeposits by our definition because they are in the body and they are physically captured if not concentrated by body fluid and/or tissue.

### 3b) Biodeposit Not Requiring A Bodily Environment:

An example of this would be a biofilm forming in a stagnant drinking-water line or fixture, another would be bacteria growing in food-processing equipment. Any bacteria growing in/on these films or in the water itself would also comprise biodeposits or pathogens not requiring a bodily environment for their formation.

### 4) Flow Plugging, Pressure Equalization Plugging, Diffusion Plugging, Current/Voltage Plugging (Some Forms of Undesired Plugging to be Addressed by the Invention)

### 4a) Flow Plugging:

An at-least temporary degrading or stoppage of a desirable flow property or flowability of one or more natural or man-made lumens, capillaries, flow channels, flow paths, membranes, apertures or orifices. To be specific, a flow rate mass-transport vector for a mobile species is at least reduced for a given available driving pressure, driving-potential or capillarity at at least one point in space and time. Examples include an excessive biofilm thickness in a catheter flow-lumen causing blood flow-blockage or flow-inhibition in the catheter lumen, an infected mass growing on an underlying seed biofilm whose physical bulk is blocking or flow-inhibiting a urine-draining Foley catheter orifice, cellular fibrous or particulate debris plugging or flow-inhibiting a urine or cerebrospinal fluid catheter orifice, precipitated insulin crystals plugging or flow-inhibiting an insulin-delivery cannula, or lipohypertrophies in patient tissues caused by delivery of insulin from a percutaneous cannula, the lipohypertrophies blocking flow of the insulin deeper or laterally into the tissue such that it cannot easily and desirably move away from the insulin-delivery cannula. By pressure, for example, we mean any manner of imposed or self-acting hydraulic or pneumatic pressure as by any type of pump, gravity-induced pressure, capillary-action related wetting, nonwetting or dewetting pressures, patient-induced perfusion or motion-caused pressures, osmotic pressure, electro-osmotic pressure, iontophoretic pressures, electrowetting/dewetting induced flow (or wetting) pressure, electrophoretic flow, ultrasound induced streaming pressure and concentration-gradient “pressure” which is the result of a concentration gradient of a species driving diffusion or mass transport of the species in a direction of lower concentration. Note that we quite reasonably define a diffusive flux as a flow. The term is applicable to device-plugging and anatomical-flow plugging. The flow may be of an entire medium or only of one or more constituents of that medium.

### 4b) Pressure Equalization Plugging:

Simply put, a desired pressure or pressure change cannot be desirably equilibrated or communicated across or through a plugged region when it is desired to do so. The plugging again may be in a bodily material such as tissue or in a device or other perturbing material, article or device. Note that in a device or apparatus designed to communicate or pass only a pressure (or pressure delta) across an orifice or down a liquid or gas-filled lumen, one can do so with negligible net flow especially assuming minimal or no compressibility of the flowable medium such as urine or blood. However the flow is not absolutely zero even in that case-and a plug can interfere even with this miniscule flow. The term is applicable to device plugging and anatomical-flow plugging. Plugging may be absolute (zero pressure communication) or partial (pressure equalization is too slow or incomplete). Devices which are designed to sense pressure frequently need to allow rapid pressure-equalization but not necessarily appreciable massflow.

### 4c) Diffusion Plugging:

In or adjacent a bodily tissue/fluid, perturbing material, article or device wherein atomic, ionic or molecular diffusion is to take place, a plug can block or interfere with that diffusive transport as by physical blockage or reactive destruction or tying-up of the desirably diffusing species. Such diffusive plugging will usually be found in exchange-membranes wherein the membrane is designed to (normally) allow cross-diffusion of one or more diffusable species. The term is applicable to device plugging and anatomical-flow plugging. A plugged electrochemical sensor, such as a percutaneous sensor for glucose measurement, can be plugged from desirably diffusing species which need to contribute to the intended electrochemical reaction and detection current. The main difference from flow-plugging is that the mobile species are typically smaller and the “pressure” driving the mobility may be a concentration gradient or potential gradient (as for electrophoretic, iontophoretic, osmotic or electroosmotic diffusion).

### 4d) Current, Voltage or Flux Plugging:

The interference with, disruption or blockage of a desirable electrical current, electrical voltage gradient, ion-current, ion-potential, ion-concentration gradient, energy-flux or particle-flux (e.g., propagating light, ultrasonic energy, electromagnetic energy, nuclear particles, charge carriers) by plugging, fouling or pathogenic material. The term is applicable to device or perturbing material/article plugging and anatomical plugging. Plugging may take place locally or regionally, over a small area or a large area. In the case of an ion or other exchange-membrane, some or all of the entire membrane area may get plugged. Plugging may be transitory or permanent. It may also be selective as for a species “A” being blocked while a second species “B” is not blocked. Desired normal unplugged flows or diffusions may be in one, both or multiple directions and may involve one or more mobile species. Note that we include in this category the disruption of applied electrical or magnetic fields-such as fields needed by sensors for their proper functioning. With respect to blocked or inhibited ion currents, we explicitly include ionic currents in nerves, muscles, neural cells, neurons and axons.

### 5) Pathogen

Any biologic, cellular, genetic, non-biologic, toxic or potentially harmful species of an organic or inorganic nature which can or might cause harm at some population or concentration level in any of its inactive, dormant, neutral, inert, active, ionized, polarized, unbound, bound, reactive, reacted, enabled, evolved or cultured states in at least one person. We include natural and man-made species. Examples could include gram-negative and gram-positive bacteria, acid-fast bacteria, E-coli, staphylococcus aureus, pseudomonasaeruginosa, MRSA organisms, pneumonia virus, prions such as infectious prions and man-made anthrax. Others include mildews, fuigi, molds and yeasts which themselves, or constituents thereof, may be harmful depending on type. Obvious additional pathogens include many viruses or any type of harmful microbe, germ or genetic constituent. We also include as pathogens, natural and man-made genetic materials and constituents and nanoparticles which would or could result in undesirable mutations or genetic consequences in or for target cells or random cells in a given setting. Pathogens might be inactive or otherwise dormant for a period before becoming active (regardless of how) and doing their harm. Harmful includes anything that causes immediate or eventual undesired cellular, tissue or body-fluid changes or body-function changes. By “undesired” we mean having or possibly having negative or non-optimal health consequences for at least one living being. A heavy toxic metal is an example of a nonliving toxic entity that we consider a pathogen. A deposit such as a biofilm which is known to host pathogenic bacteria can be considered harmful because of its pathogenic potential in addition to any bulk flow-blockage issues that it itself may cause. A pathogen can be harmful simply because even if only one or a small number exists it may later react and/or multiply to a harmful level or state.

### 6) Antipathogenic

A material, material surface treatment, material bulk-compositional treatment, a material composition, a drug, a biological or genetic entity, a process, a device or an exposure to radiation, chemicals or energy which is capable-of or actually-does avoid, suppress, minimize, inhibit, reduce, disable or kill at least one pathogenic species, directly or indirectly. An antibacterial at least attacks or inhibits some bacterial pathogens. An antiviral at least attacks or inhibits some viral pathogens. A drug (or beneficial bacterial species) which sponges-up or ties-up toxic metal contamination in a human body is also herein regarded as antipathogenic presuming the benefit of deactivating or removing some of the toxic material outweighs any practical negative side-effects of the beneficial bacteria. Sterilants, some silver, copper and silver/copper ionic species, UV light and ozone are antipathogenic as is ultrasonic cavitation and even lower-intensity ultrasound and pulsed and CW electrical-fields which have been found to disrupt external cell membranes and/or interior-cell entities and membranes. All of these kill, harm, inhibit or prevent the cell-division-of or otherwise disable pathogens to various degrees. Similarly antimicrobials, fungicides, sterilants and germicidals are considered herein to be antipathogenic. It will be recognized that many antipathogenics can also kill healthy cells (e.g., radiation, ultrasound cavitation, ozone, UV) if delivered in too high a dose, purposely or accidentally, to healthy cells. Our inventive use herein of highly localized pulsed electric fields assures minimal healthy tissue destruction.

### 7) Pressure

Any type of mechanical, fluidic, hydraulic, pneumatic (gaseous), capillary, wetting or shockwave induced stress present or applied in one or more axes or directions delivered or generated by any means upon or in any material, whether the material is flowable, deformable, compressible or not. Typical pressure application waveforms will be alternating in positive and negative pressure (bipolar) or unipolar comprised of compressional or tensile longitudinal waves or shear waves. Pressure waveforms may be at any frequency from 0 hertz (DC static pressure) to Megahertz and higher for cyclic, pulsed, harmonic, non-harmonic or non-periodic pressures. Pressure waveforms may also be single-pulsed or have extended waveforms that are not harmonic or periodic in nature or may even be random in nature. Pressurization events or waveforms may have any magnitude of applied stress or pressure (load/area) and may have any temporal period or duty cycle. “Pressurization” Thus, includes applying or causing a negative gage-pressure or absolute-pressure otherwise known as a gage-vacuum or absolute-vacuum. Pressure waveforms are typically applied by pumps, volume-displacing mechanisms, constrained thermal expansion/contraction effects, transducers of various sorts, mechanical deformations, shock-waves, the application of force-fields (e.g., gravity, acceleration, magnetic forces, etc.) and the application of mechanical shocks as by impact. Ultrasonic pressures are those developed by ultrasound waves. We define sonic as 0-25 kHz (generally in the human hearing range) and 25 kHz and above into the megahertz (and above) regimes as ultrasonic waves or ultrasound. Typically, a pressure will be present in a region or volume of material-albeit that volume might occasionally be exceedingly small or very large. Flowable materials typically support static or pseudostatic triaxial uniform or near-uniform pressures whereas non-flowable or less-flowable materials can also or instead support uniaxial static or pseudostatic stresses and shear stresses because those materials have shear strengths. Pressures may vary over long periods or very short periods-or be constantly applied. Shock waves-as applied by ultrasound for example-can cause non-equilibrium pressure (stress) states in the mediums through which the shock waves or ultrasound waves pass. Note that such states will typically be short-term or dynamic in nature. These pressure waves are typically not static or even pseudostatic at a given point.

Our invention utilizes acoustic (non-cavitating and/or cavitating) waves as one mechanism of dealing with pathogens and potentially fouling or pathogen-breeding deposits. We define acoustic-energy globally as including all manner of applied infrasonic, sonic and ultrasonic propagating pressure waves as well as static and pseudostatic pressurizations or pressure-states. Pressure may be in equilibrium or non-equilibrium, changing or constant. Pressure may have both AC and DC components. By our definition above, an applied vibration is an applied acoustic energy. Our earlier mentioned electrophoretic, electroosmotic, electrowetting and electrorheological flows are driven by electrical field potentials which can be thought of as “pressures” as well. Any of those can be pulsed or constantly applied.

### 8) Lipohypertrophy

A fat-pad is typically comprised of fibrous and fatty tissue. These can be caused as by the growth-factor effect of percutaneously delivered insulin-the result being a fat pad growing at the percutaneous cannula insertion site which can impede further insulin inflow (i.e. via tissue and/or cannula liquid-flow plugging). This phenomenon can cause a patient not to reuse that particular stick-point for an extended period-if ever again. This particular flow blockage might be one or both of fluid-flow or diffusive blockage. Note that this particular case is a form of plugging caused by a reaction of a patients tissue/fluid to a device and/or to its delivered medication.

### 9) Redness, Irritation, Infection, Swelling

Such as at a drug-delivery cannula site, an example of this being an insulin delivery-cannula percutaneously delivering insulin which also contains an insulin preservative. A patient may become reddened, irritated, swollen or even infected due to his/her allergy to the insulin itself or due to an allergy to the insulin's own self-contained preservative. A similar reaction may be caused simply by a foreign body (cannula material) reaction. These can cause the patient not to reuse that particular needle or cannula stick-point for an extended period-if ever again. The very act of needle-sticking and/or skin-contact of any adhesive component that is part of a percutaneous cannula may also or alternatively cause swelling, skin irritations and/or redness. All such reactively formed tissues and fluids are herein regarded as undesirable deposits or fouling which can be addressed using the invention. Note that this behavior is desirable for an unplanned or unwanted puncture wound.

### 10) Orifice (Prior Art)

A hole, aperture or flowpath through or along which a flowable or propagatable medium such as blood, urine, cerebrospinal fluid, a drug, insulin, water, gas, air, interstitial fluid or other useful species is passed or flowed. These orifices are typically single holes (or collections of holes) in the walls of catheters and are typically punched or laser-drilled. They are easily clogged by particulates and are difficult to keep free of deposits.

### 11) Permeable or Virtual Orifice/Membranes

These are flow-permeable or diffusive-permeable orifices comprised of a region of two or more typically internally cross-connected flowpaths. A permeable orifice made from sintered polymeric microparticles would serve as an example-permeable pore space being left between said particles during the polymer-powder sintering process.

Presuming the permeable pore space is wettable by a fluid or medium to be passed therethrough then self-wetting will occur and the fluid may be continuously passed there through as by any continued pressure gradient. Presuming the permeable pore space is poorly wettable or unwettable the fluid passage or entry will require application of a forced-ingress Laplacian pressure. This pressure is higher for increasingly unwettable pore space and for increasingly small pore space.

Because an unwettable or poorly wettable permeable orifice requires forced fluid ingress we have inventively utilized it to provide an orifice which naturally desires to be liquid-evacuated such that pathogens and deposits cannot grow and/or survive therein during such unintruded periods. The liquid can be readily evacuated as by allowing it to retreat from the permeated material to an adjacent reservoir after intrusion pressure is removed or reduced.

We further recognized that permeable orifices (or permeable lumens) provide a unique situation wherein undesirable constituents present in a passing fluid can be efficiently exposed to antipathogenic treatments. Note that a fluid passing through a tortuous permeable-path orifice will force undesired fluid constituents (such as pathogens) to be exposed for longer periods and at more angles relative to the permeable orifice as a whole. Thus, if the permeable orifice is arranged to incorporate drugs, chemicals, electrical fields or radiation inside of it or across it, the passing constituents will be thoroughly exposed-to and interact with such drugs, chemicals, fields or radiation. The source of such antipathogenic drugs, chemicals or radiation might be the permeable material itself, a coating on/in a permeable material surface (interior or exterior) or surface(s) of the permeable material, or an external source outside of the permeable orifice itself. Inventors also utilize the permeable orifices to support electrodes with which electric fields and/or currents can be applied to passing mediums such as to kill pathogens in the medium or to kill pathogens potentially growing in the permeable material itself. We note that such electrodes can be closely spaced and thereby offer very high voltage gradients. Such electrodes may easily be fabricated of non-corrosive metals such as titanium, gold or platinum.

Note that we can pulse flow or slowly pass a fluid through such a permeable orifice such that while a given bolus of fluid is resident in said permeable orifice it and any foulants it holds receives a time-extended or at least multiangular exposure or treatment time by the antifouling electrodes, chemicals, drugs or radiation.

Finally, by permeable or virtual orifice we include not only localized orifices but also permeable or diffusive membranes which frequently extend for significant lateral distances. In this manner we can clean or keep-clean such membranes. Such membranes are used in dialysis and in seawater desalinization for example. Included in our scope of permeable orifice and membrane cleaning is applying our cleaning mechanisms during a temporary reverse-flow or flushing step after which normal flow or osmosis may continue. In that manner the deposited fouling matter can't block operation or cleaning.

We again explicitly note the special case of zero or near-zero flow orifices-most applicable to our inventive permeable orifices below. These include pressure-communication orifices or membranes (e.g., liquid to liquid, gas to gas, liquid to gas) and orifices and membranes across or through whose contained parent medium is passed electrical current, ion current, a potential or potential gradient of some sort (e.g., voltage, concentration), diffusion of a species or charge carrier, or passage of a form of energy such as RF, optical, laser or ultrasonic energy. Such useful orifices, apertures, membranes or energy-windows can often operate with little or no mass flow of the parent-medium if required to do so or if that is beneficial. as it would be for a blood pressure sensor, for example.

Permeable or virtual orifices and membranes actually have several desirable traits and we collectively list some of these here:
a) They are not easily plugged due to their physical filtering properties making them immune to one or a few conventional particles, globs, precipitates or fibrils.
b) A large sized virtual orifice does not compromise structural integrity of the catheter or parent-body as it can have its own rigidity unlike a prior-art hole-type orifice, yet it can assure a minimal flow for an extended period.
c) They do not easily allow biofilm growth in or on it because the permeable material it is comprised of, inherently has, or is treated to have biofilm suppression properties. It may also have a surface or internal pore size which is size-limiting to invading or ingrowing cell types.
d) They can be arranged to have an enabling Laplacian pressure only above which any inward or outward flow can occur-thereby optionally isolating, for periods of desired length, external bodily tissues and fluids from internal flow lumens.
e) They allow for through flow or intermittent flow stoppage inside them for purposes of flowable-medium exposure or extended exposure to treatments such as antipathogenic treatments. Such exposures may be from internally or externally provided chemical, drug, radiation, electrical voltage or current or other energy exposure.
f) They can be made flexible such that the flexing motion serves a useful function such as an electrical contact or fluidic valving function or provides orifice/membrane distortions or stretching useful to orifice/membrane operation or cleaning. Switching could be done as by electroding one of the contacting flexing surfaces. Note that in the case of a distortable orifice, distortable as by overpressuring, one could use such distortion to forcefully clean the orifice in a forward or backward flow event.
g) They can be arranged to have an electrode(s) across some or all of its flow path(s) to impose or induce a field, bias, energy flux or electrical/ion current useful for several purposes including our inventive antifoulant ones. Undesired flows, such as of specific species or pathogens, may thereby be electrically prevented or stopped at flow or non-flow locations. Note that an electrode pair could be arranged either along the flow direction or normal to it. We include in our scope the employment of our applied electrical fields for the purposes of electrophoretic, iontophoretic, and electroosmotic pumping or pressurization as already mentioned.
h) They can serve as an isolation window, aperture or screen through which to “remotely” sense a parameter, to pass energy or radiation, or to communicate potential or concentration. By isolation we mean at least one species or at least one energy of a certain parameter (e.g., wavelength) is excluded from passage. Thus, for example, one could sample gases emitted by a fluid on the other side without direct contact with the fluid.
i) They can force passing flows in extremely close proximity to antipathogenics, drugs or other biological or non-biological agents which are deposited on, in or even comprise the permeable virtual orifice or membrane material or which are a constituent of said material. This guarantee of intimacy can assure complete exposure with a single flow-pass.
j) They can act as a physical, electrostatic, electrophoretic or iontophoretic filter for ingoing/outgoing flows.
k) It can be selectively cleaned or sterilized as by application of a radiating energy, targeted heating or pulsatile flushing with or without the assistance of ultrasonic or acoustic excitation.
l) If its permeable passages are selectively activated (physically created), as they would be by temporarily opening them up with an applied fluid pressure, they can then be later closed when not in use. This we refer to as temporary permeability or applied variable permeability. Such a material with pressure-created permeability may or may not be Laplacian.

In the case of flexible or deformable membranes such as dialysis membranes or desalinization membranes one can apply the inventive ingressing Laplacian pressure in support of antifouling without tearing or ripping the membrane as by supporting the membrane on a rigid permeable ceramic or glass substrate. That substrate could have larger pores if desired as it can still serve as a rigid base for the overlying pressurized flexible polymeric membrane. Within our inventive scope though is the purposeful deformation and/or distension of membranes below a ripping threshold since that can allow for some unsupported pressure application and can actually reduce the Laplacian threshold pressure via tensional enlargement of the interpore spaces.

### 12) Lumen

### 12a) Natural Lumen:

Any natural passage in a human or animal body such as a vein, artery, capillary-bed, sinus, intestine, liquid cavity such as a cardiac chamber, a neural or spinal CSF ventricle or a gas cavity such as a lung space, bowel space or sinus cavity. A virtual natural lumen, such as the virtual space between normally juxtaposed organ interfaces, may be opened up or accessed by gently pushing a medical device, tool or gloved finger through the unattached or weakly bound interface. We consider this as a normally-closed natural lumen or virtual space.

### 12b) Man-Made Lumen:

Typically, a medical device lumen such as a catheter or cannula flow-passage for a bodily fluid, drug, genetic-therapeutic or nutritional material. Lumens may also comprise the flow passages in the piping of a food-processing factory, pharmaceutical plant, pool or water-distribution system. In any event, a man-made lumen will most often be made for a purpose such as the flow, transport, analysis, storage, processing or pressure-exchange of a useful medium. By flow we mean any manner of mass transport of at least one constituent of the medium including fluid flow and gaseous, ionic or vapor diffusion. Typically, a flowable medium will flow to or from an orifice (prior art or inventive types) through one or more lumens. However there are some cases wherein an orifice itself can act as a flowable-medium reservoir such that a connected flow-lumen is not necessarily required. (e.g., a drug depot comprised of a drug in a permeable reservoir-the permeable reservoir surface acting as a distribution orifice/depot or distribution surface). Man-made lumens certainly include pipes, ducts, conduits, tubes, wicks of all types, man-made capillaries, open flow-channels, hoses and hypodermic needles and microfluidic flow-features. Man-made lumens may have one or more flow-passages or conduits-including the case wherein the flow-conduit(s) is a permeable material such as the permeable core of an otherwise non-permeable tube.

Per our above discussions we include in man-made lumens the “flow” of charge carriers, ions, photons, phonons etc along any defined path therefore electrical leads, optical fibers and acoustic waveguides, whether insulated/isolated or not from their immediate surroundings, are herein also considered to also be man-made lumens that pass transportables. Note that a copper conductive wire is a lumen for electrons by our broad definition.

### 13) Flow

Any mass-flux, mass-transport, diffusive flux, massless flux or current of a flowable or diffusable medium or species such as of ions, atoms, molecules or other charge-carriers such as electrons or holes. Mass transfer per entity depends on the mobile or radiative species or entity involved, but charge carriers such as electrons and holes will have negligible to zero mass transfer as will photons and phonons. Flow may be periodic, constant, unidirectional, bidirectional or multidirectional. Flow may be forced or unforced. Forced flows include those urged by pumps, conventional heads of pressure, acceleration, gravity, electrowetting, ion-currents, electric fields, magnetic fields and acoustic-streaming pressure. Unforced flows include those caused by capillary action, wettability phenomena and concentration gradients. In order to flow, a flowable species might even undergo a phase-change such as melting from solid to liquid or sublimation from solid to gas/vapor. A macroscopic flow might be measured in grams/second and a smaller or microscopic flow may be measured in atoms or ions/second, for example. We include, as further forms of flow, the flow of ions (which also comprises an electrical current) and the flow, migration or passage of neutral or charged entities such as electrons, holes, neutrons, protons, molecules, biological constituents, nuclear particles, alpha particles and ions. A flow usually involves some mass transport, albeit possibly miniscule, when the mobile entities are atoms, ions or molecules. For mobile electrons, holes, photons, phonons etc the flow may be measured in electrical current or in energy transferred despite minimal or zero mass transfer. A flow or flux may be steady, changing or even random. Thus, “flow”, for example, includes any of ion-diffusion through a cell membrane, arterial blood flow through an artery, the movement of an atomic or ionic species along a concentration gradient and the movement of a charged or magnetic species along an electrical or magnetic gradient. A diffusive flow may have a net-zero transfer rate across a given interface because leftward and rightward nonzero fluxes are in time-averaged equilibrium with respect to concentrations on each side. A flow of a charge-carrier implies also a flow of current. A flow of radiative energy such as infrared energy is usually called a flux as it has no measurable mass transport associated with it but herein we shall also refer to such radiative energy transfer as either a flow or flux interchangeably. Again, rather than significant mass transfer, those involve charge or energy transfer.

### 14) Inventive Defouling or Antipathogenic Mechanism Examples

a) Application of a first pulsed electrical field to kill pathogens and/or biodeposited cellular matter followed by an ultrasonic shedding and flushing of the resulting dead cellular matter. (This is at least additive and likely synergistic if the dead cells are more easily cleanable acoustically than live cells.) The electrical field pulses may be short enough to cause internal cell damage and/or external cell membrane damage per the above discussions and/or electroporation or oxidation as by passage of a longer sustained current or cumulative current over time.

b) Application of a fluidic pressure pulse to cause macroscopic unplugging of a lumen (if any plugging exists) followed by a pulsed electrical field to kill any remaining surface-attached or near-surface pathogens and/or cellular deposits. (This is at least additive and might also be synergistic if, because some deposits were already removed by pressure flushing, a more damaging (higher) voltage gradient is applied to the fewer remaining cells to be killed.)

c) Use of a permeable orifice having an interior drug coating to kill resident or passing pathogens or cellular matter followed by or at the same time with an applied pulsed electrical field to kill the same or different pathogens or cellular matter. (This is at least additive but likely also synergistic because each mechanism inflicts damage that makes the other mechanism even more effective.)

d) Application of a first electric field pulse of a first longer duration to cause eletroporation of some pathogenic or deposited cells and a second application with a different energy or shorter pulse duration to cause intracellular (inside the cell) apoptosis, the pulses being delivered sequentially, simultaneously or in an interleaved manner. (This is at least additive but likely also synergistic if one type of damage invites more easy production or maintenance of the other type of damage.) For instance, outer membrane destruction exposes the interior membrane structures to more damage.

e) Use of a known biofilm-inhibiting material such as Teflon®, permeable or not, and the use of pulsed applied electrical fields. These may act on pathogens or deposits in the same or different regions. (If the application regions are different, then it is additive, but if they are the same, then the poor wettability of the Teflon® might render surface pathogens and/or cells more susceptible to electric-field induced damage.)

f) Scheduled or feedback-based application of short nanosecond regime electric field pulses at the tip of a drug delivery or drainage catheter which at least cause intracellular or cell-internal damage or apoptosis of at least some undesirable pathogens or deposits. (This is likely a single mechanism.)

g) Flow of a bodily fluid through a flowpath wherein the flowable medium first passes through an electrical field then, downstream, passes through a drug-infused permeable filter or orifice (This is at least additive but possibly synergistic if the electric field makes pathogens or undesirable cells more susceptible to drug interaction.)

h) Combined simultaneous exposure to a short applied pulsed electrical field and acoustical energy. (This is almost certainly synergistic in that one is attacking both external cellular and internal cellular membranes simultaneously.) Note that in this example one could utilize cell-interior constituents not normally viably available outside the cell membrane to interact with other cells or other cell contents. Thus, one could cause apoptosis of more than one cell including as by exposure to adjacent treated cells or adjacent more apoptosis amenable cells. In this manner, cancer cells could be surrounded and penetrated with apoptosis inducing constituents release by or produced by one or more other distant or nearby treated cells.

It is important to note that while the invention will deliver antipathogenic and/or antifouling capabilities, one may choose to also employ other beneficial drugs or medicaments for nonpathogenic, pathogenic, antifouling or fouling-reversal purposes. We include fouling-reversal or foulant cleaning as “antifouling” behavior. Such other drugs might, for example, include anticlotting, antiscarring or antiswelling drugs. Such a drug might also be a systemic antipathogenic possibly operating additively or synergistically with a foulable device-contained antipathogen or antipathogenic mechanism(s) and/or drug(s). We included above a list of several known antiswelling, antifibrous and antiscarring drugs. Such agents may be used systemically or locally to further enhance benefits to the patient or subject whose safety is to be assured. Thus, the invention may be favorably utilized together with systemically administered drugs or medicaments which may be different or the same drugs as those utilized locally in/at the foulable material, article or device itself. Included in the inventive scope is the case wherein a foulable device, article, or material defouling mechanism includes a device-local drug, said drug, over a period, being taken up systemically from the local device/material drug reservoir or depot. After being taken up systemically it may be arranged, or not, to still have remaining patient-beneficial activity.

It will be noted that the two inventive defouling and/or antipathogenic mechanisms and mechanism combinations utilizing one or more of them will typically act in, on or near a device, article or equipment item or on a medium such as drinkable water or milk processed or stored thereby. The device, article or equipment item may or may not be juxtaposed-to or inside a living being (as for a surgical application) and may simply be part of an inanimate food or drug-processing plant. In this vein we stress that the source of driving for our inventive mechanism(s) may be in situ or external. For example an ultrasonic mechanism can be provided by a bodily in situ or body externally applied transducer. A drug can be provided systemically and/or in an in-site permeable orifice for example. It is the mechanisms and mechanism combinations we claim regardless of their manner of delivery or application.

### Discussion of the Figures:

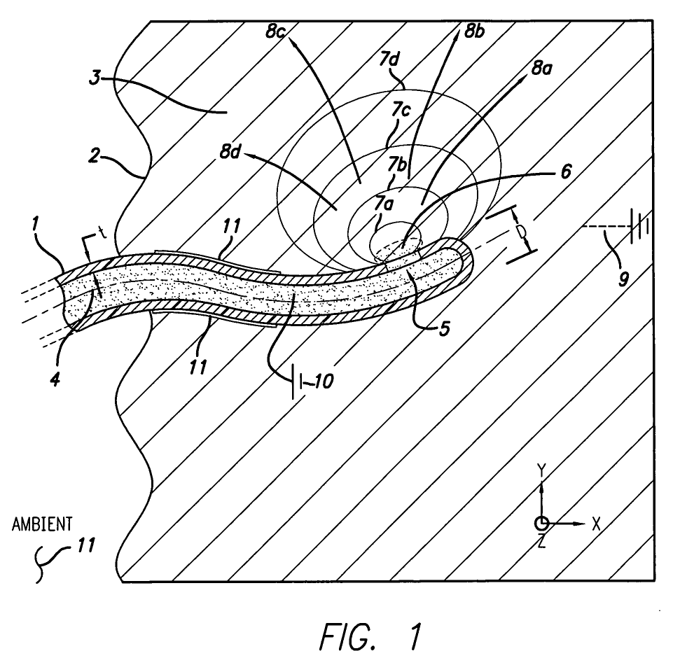

Starting with FIG. 1, we see a depiction of a flexible catheter 1 inserted into a human body 3 through an incision, natural lumen or natural cavity. The tip region is surrounded by a liquid or fluid film naturally or via inward delivery thereof through the catheter.

The catheter has a diameter D and a wall thickness t. The catheter 1 is depicted being filled with a liquid electrolyte or electrically conductive liquid 4. The catheter 1 has an orifice 5 allowing flow communication between the catheter interior and the surrounding tissue/fluid 3. An XYZ reference system is also shown.

FIG. 1 depicts the implementation of the preferably-pulsed electrical cleaning, defouling or antipathogenic capability of the invention. Essentially, the electrically conductive fluid 4 in the catheter is pulsed with an electric potential waveform by a power supply or energy source 10 shown schematically. The pulsing source would ideally be outside the body. The liquid 4 acts as an electrode, causing a momentary electrical current to flow out of the orifice 5 into the body 3. We show these momentary current paths as 8a, 8b, 8c, and 8d spreading apart away from the orifice 5 with the familiar approximate 1/r³ behavior for a 3-D point source. We also show momentary electrical isopotential lines 7a, 7b, 7c and 7d. As the reader will appreciate, the current density and field drop off rapidly with distance from the orifice for such a point source at orifice 5. The pulsed current is sunk into a virtual capacitive body electrode indicated by 9. Typically, this capacitance will be formed by bodily tissue/fluid 3 in the region into which the current-pulse flows, which may primarily comprise a thin small region of bodily tissue/fluid in/at/outside the orifice. Assuming no second body-attached or inserted electrode is utilized, then the pulsed current is that which charges the tissue capacitance as provided by the very short pulses in the nanosecond(s) regime as described earlier. The needed tissue/body fluid conductivity either already exists or is provided or enhanced as by forcing a conductive fluid 4 such as saline out of the orifice 5 to saturate into or wet the tissue 3. The conductive fluid might include or be a drug, medicament, saline, plasma, nutrient or even a fluid being drained from the body (such as urine). If it is an ingoing fluid it may need to be doped with a conductive or ionic species to make it conductive, such as a salt or metal ionic species.

By delivering nanosecond(s) regime waveforms using a power supply or pulser 10, shown schematically, one will obtain the necessary high electric fields particularly at and near the orifice 5 such as are known to be capable of altering or destroying cell-interior entities as described earlier and by Beebe. We emphasize that ideally the pulses are applied voltage pulses which induce the momentary capacitive current 8 and driving field 7; however, applied current current-limited pulses may also be utilized if they can be conveniently generated. Given that in FIG. 1 we apply our altering or disrupting waveforms at the orifice 5, then any cellular matter at that location of high voltage gradient will be so-altered or disrupted. We specifically include cellular matter anywhere in the general vicinity of the orifice, whether outside, in the orifice, or inside the catheter near the orifice. Presuming the orifice 5 has a small diameter relative to the catheter interior diameter, then the current-density and field strength in the orifice 5 will be higher than that in the fluid interior of the catheter 1 itself. Of course if the pulses or waveforms applied are intense enough, then one can reach cell altering or disrupting conditions along the catheter lumen interior as well, thereby affording catheter interior defouling.

The catheter 1 of FIG. 1 is depicted as also having a surface condition or treatment 11 along a portion of its outer diameter. For example, surface conditioning 11 may be an antifouling drug coating or impregnation, a silver-based antifouling coating or impregnation, or a low wettability Teflon® material that inhibits biofilms, in which case the Teflon® might constitute more than just the surface layer. Thus, in FIG. 1 we have two example mechanisms working to defoul or keep the catheter 1 and its bodily environs defouled-namely pulse-based cell-interior disruption and an antifouling surface-engineered portion 11. Note that in this case these two mechanisms operate on foulants at different locations. In other cases two or more mechanisms will operate on foulants at the same location.

The orifice 5 may comprise one or more flow passages, including orifices that simply communicate pressure or potentials as described earlier. The orifice may also be permeable in nature, Laplacian or not. The catheter device 1 of FIG. 1 might be utilized to one or both of deliver a medium to the body or remove (e.g., drain) a medium from the body or both. In any event, to support the electrical pulsing, the electrical current will have to flow along the catheter and then out the tip. Ideally, for simplicity, the fluid being delivered to or drained from the patient ⅔ is itself sufficiently conductive, as is saline or urine, for example. In that case, that liquid itself can act as the application electrode. However within our intended scope is any implementation wherein a field or current is applied to undesirable pathogens, biodeposits or cellular matter, regardless of whether the fluid along the length of a catheter interior acted as a delivery electrode. In other words, we could have alternatively delivered our voltage pulse to the orifice 5 via a conventional electrode or wire (not shown) and still have the desired effect at the orifice. The simplest and preferred arrangement as shown is an electrically insulating polymer-based catheter body 1 that contains an ionically electrically conductive flowable liquid. In this manner, one gets the desired point source-driven cleaning at the orifice with an inexpensive to-make catheter, We show the pulser circuit or component as 10. Of course in a real system, this pulser will likely be outside the body in a console or in an ambulatory or wearable pulser device. Beebe and others have described high voltage pulsers such as nanosecond-range spark-discharge pulsers utilizing electrical transmission lines. Finally, we emphasize that if one utilizes a flowable fluid or medium such as 4 as an electrode anywhere, then that liquid might be a bodily fluid, a medicament, or even a temporarily introduced liquid introduced for occasional pulsed cleaning. In that manner, any flow of liquid into or out of the orifice 5 will depend on whether such flow is a medicament, for example, or a cleaning fluid such as saline. We emphasize that to achieve the pulse based electrical cleaning, no fluid flow is required for that purpose, although flow can be allowed if it is convenient. We also note that our pulser means can also treat moving foulants or pathogens, including those moving in a flow through the orifice. In this manner, the pulsed orifice can act to process flowables or materials that are passed through or nearby it, thereby extending its benefit beyond the 1/r³ behavior, because treated entities will move beyond their locations whereat they were treated. Finally, we remind the reader that “pulsing” might include short pulses attacking cell interior contents and/or longer pulses attacking cell external membranes and that such pulses or pulse trains might have a net averaged directional charge transfer or not. By fluid 4 being in the lumen we mean it is likely filling the lumen (shown); however, we include the case wherein it simply wets the lumen wall but still provides an electrical path.

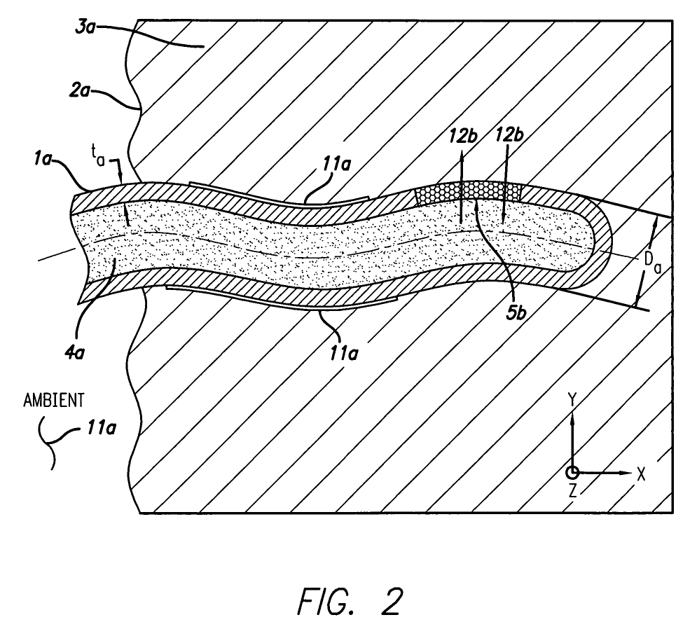

Moving now to FIG. 2 we also see a catheter or other tubular-based elongated medical implement such as a scope 1a. In this case, the diameter Dₐ is shown larger than that in FIG. 1 and the wall thickness is also thicker and shown as tₐ. We depict a fluid 4a in the catheter/scope 1a interior that may or may not be conductive for our depicted Laplacian orifice (or membrane) purpose here. A permeable Laplacian orifice 5b (or membrane) is depicted in the wall of the catheter/scope 1a near the scope tip. As described earlier, a Laplacian orifice or membrane is one that can be forcefully ingressed with a flowable liquid or media or other ingressing species by applying an ingress threshold pressure differential across it otherwise known as the “Laplacian Pressure”. This pressure differential is actually a pressure delta or gradient applied across the orifice or membrane 5b. Depending on the direction of the applied pressure gradient, the ingressing action or progression will proceed toward the lower pressure side. For example, if the catheter/scope 1a interior is pressurized positively with respect to the body tissue 3a, then fluid 4a will ingress the Laplacian orifice 5b above the Laplacian threshold pressure in an outwards (toward the tissue 3a direction as indicated, for example, by an outward ingress and flow 12a. Likewise, if the catheter/scope 1a interior liquid 4a is held at a relatively lower pressure than the surrounding tissue/bodily fluid 3a, then an inward ingressing flow 12b will take place. As taught earlier, because we can selectively apply or unapply the enabling threshold pressure differential, we can likewise apply or reverse an ingress of fluid. We can create a dry or non-ingressed orifice 5b when we wish since a dry orifice is more resistant to fouling and pathogens. By “dry” we mean relatively free of at-least the species that behaves in the Laplacian manner. Further, we can utilize the Laplacian orifice as a valve contributing to a valving function of the device itself. As an example, the catheter/scope 1a might be a urine drainage catheter. When we apply negative pressure to the catheter interior, we can enable (switch-on) urine outflow. When we remove the negative catheter-interior pressure, the urine flow stops. If we apply a positive interior pressure now using saline instead for example, we can reverse flush the orifice if not the bladder itself with saline. The flushant could easily contain medicaments or antipathogenic constituents as well.

We note that when the Laplacian ingress pressure is removed, the formerly ingressed fluid will flow out of the orifice 5b and either ball up on its surface or flow away via any wettable or prewetted path such as backwards toward the formerly threshold-pressurized face. In some applications, it may be beneficial to provide wettability of one or both of the entry surface and exit surface but of course leave the orifice interior unwettable such that it still has a Laplacian threshold. This will help avoid problems with bubbles and pressure-threshold uniformity.

We note that some applications require a pressure-controlled drain as does drainage therapy for hydrocephalus. In this case, one could easily arrange for the moving drained fluid to be CSF or cerebrospinal fluid such as that it is being drained by an intrathecal catheter into the abdominal cavity. One could set the orifice ingress pressure (ingress from outside into the drainage catheter) at the desired maintenance pressure or maximum desired pressure of CSF. In this manner, undesirable higher CSF pressures will themselves cause orifice ingress and self-actuated pressure relief. Note that even on the urine drainage application, one might utilize this capability to automatically keep the bladder below a desired urine pressure or degree of distension.

As taught earlier, the invention might utilize conventional, permeable or permeable-Laplacian orifice(s) or membranes. Any of these, as also described earlier, could be outfitted with one or more electrodes (none shown at orifice 5b in FIG. 2). Such one or more orifice electrodes could be utilized to apply electrical fields or currents to flowables, foulants or pathogens in, passing through, or in the vicinity of the orifice(s) 5b. Such applied fields or currents might achieve several taught purposes such as i) electrophoretic pumping or separation, ii) pulsed cell-interior damage or alteration such as to pathogens or disease cells, iii) cell-exterior damage or alteration such as to pathogens or disease cells using longer pulses or continuous currents, or (iv) iontophoretic drug delivery or species manipulation. A convenient location for such a pair of electrodes would be on the inlet side and exit side of the orifice. On of these electrodes might even be provided by a virtual electrode comprising body capacitance or lumen/lumen-fluid capacitance.

We earlier grouped orifices and membranes together as they can both benefit from the invention, so we again stress here that orifice 5b might alternatively be a membrane and be a membrane which is designed, for example, to normally pass some form of electrical or chemical species selectively-such as a dialysis membrane in vivo or ex vivo. Electrodes could also be applied to such a membrane to achieve the similar taught purposes. Those electrodes, if they potentially block flow or species flux, could be patterned or porous themselves. They could be mounted such that the field gradient is parallel to the flow/flux of species or perpendicular to the flux, for example. A membrane 5b may also have a much larger size or area than an orifice 5b and may be accompanied by or fused or bonded to a backing stiffener such as a porous ceramic or glass or a metal screen.

In FIG. 2 we have shown again a treated catheter surface portion 11a. In this case, the treatment(s) is a combination of a low-wettability fluoropolymer composition (or surface coating) and a surface and/or bulk impregnated antipathogenic drug.

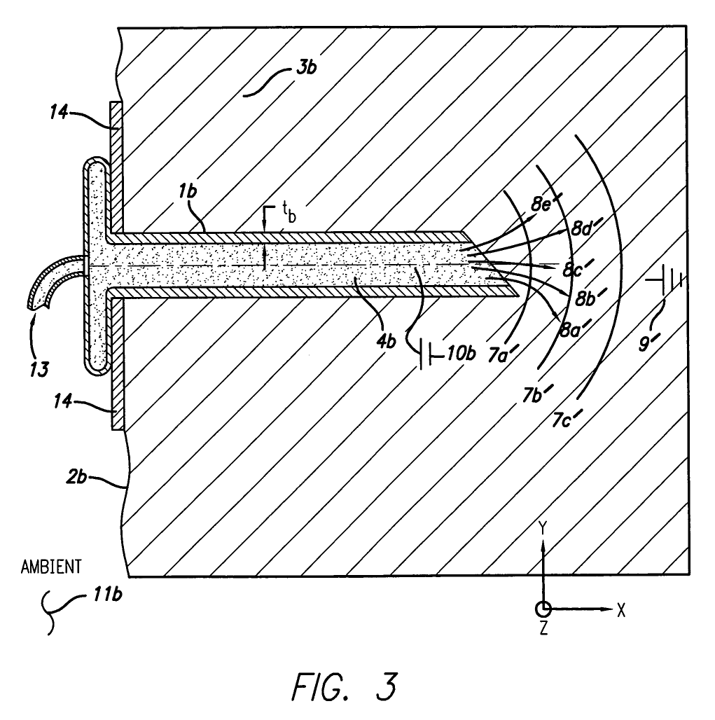

Moving now to FIG. 3 we see a Teflon® electrically insulating (nonconductive) tissue-inserted hollow needle lb used to deliver insulin to a diabetic, for example. Such hollow needles are frequently inserted with the help of an internal solid stiffener metallic needle or rod that is then removed and is therefore not shown. The insulin is the liquid 4b within needle 1b. It is treated, as and if necessary, to have an electrical conductivity such that it can preferably act as a liquid electrode for our electrical pulsing means. As with the previous pulser-related figures, we see in FIG. 3 a schematically applied potential supply as item 10b and the familiar diverging momentary current lines 8a′, 8b′, 8c′, 8d′ and 8e′ and electrical isopotential lines 7a′, 7b′ and 7c′. Again we note that pulser 10b will most likely be situated outside the body 3b and be connected to needle 1b with an electrical transmission line (not shown). We note again that if one provides an actual electrode or transmission line in the needle, then one need not rely on liquid conduction along the lumen. In any event, in order to provide our defouling or antipathogenic effect at the needle outflow tip, we want to apply our electrical gradient at least to the tip region as shown and this could instead be done as mentioned by having a conductive electrode inside the needle lumen or wall such that it can get a potential to the needle tip. The needle of FIG. 3 utilizes electrical defouling or antifouling of its tip region but it will also apply voltage gradients to the fluid in the lumen 4b. If the object is to primarily treat the tip region electrically, then one would arrange for the voltage drop along the liquid-filled lumen to be small in comparison to that at and outside of the tip. Providing a solid electrode in the needle 1b wall or inside diameter instead (not shown) would certainly assure minimal or no voltage drop along the needle 1b shaft and maximal drop at and outside the tip region.

If we had made the needle 1b of FIG. 3 out of a conductive metal such as Nitinol™ or stainless steel (not shown), then we would be applying our electrical potential to the entire needle body. This would give a 1/r³ gradient at the tip and something like a 1/r² gradient along the cylindrical needle outside surfaces. One could insulate the tip or increase its effective radius in order to suppress current from the tip if desired, such as if primarily lengthwise cylindrical gradients were desired. If one uses a conductive needle but only desires electrical treatment at the tip, then one will likely insulate the needle outer diameter to prevent or minimize current flow out of that surface. Tube 13 in FIG. 3 is a fill or feed port for insulin and likely comes from an insulin pump (not shown). Item 14 is an adhesive pad to hold the insulin needle onto the skin 2b once inserted as is the current practice for existing uncleanable needles.

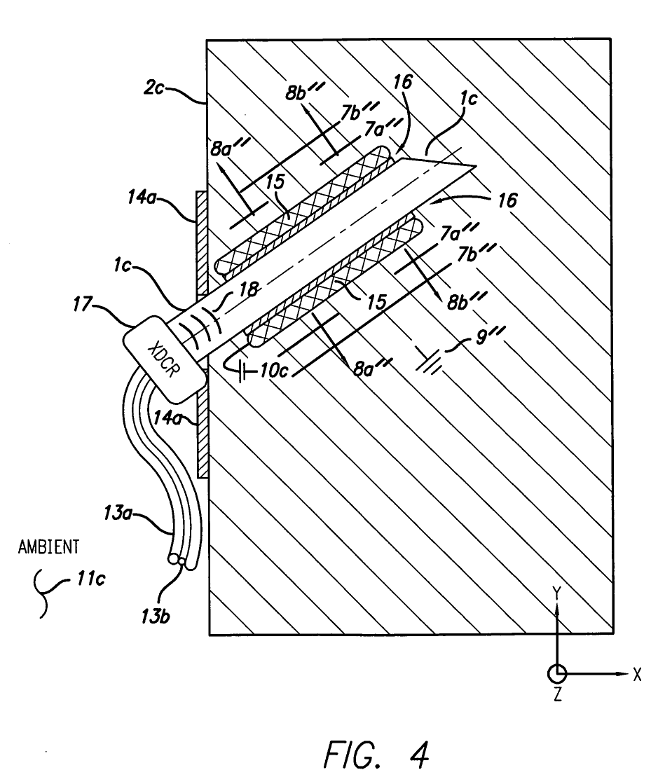

Moving now to FIG. 4 we see a tissue-inserted electrochemical-based glucose sensor 1c for use in a diabetic. These sensors have a coating 15 of a compound which chemically reacts with glucose and produces an electrical detection current which is fed to a current meter. A common problem is that the active chemical coating becomes overgrown or fouled, perhaps even infected. In FIG. 4 we provide two complimentary mechanisms for preventing such problems. The first is cylindrical conductive electrode 16 utilized to apply our pulsed voltages across the overlying cylindrical chemical coating 15 and the tissue just beyond it. In doing this, we create cylindrical isopotential lines (cylindrical surfaces) 7a″ and 7b″ as well as momentary current lines 8a″ and 8b″. Note that the dielectric constants of the coating and tissue/fluid involved will determine the relative voltage drops across the coating and into the near tissue. Within our scope is the adjustment of the coating dielectric constant and/or conductivity to optimize the defouling voltage application while still providing the desired glucose sensing current. Again. for short pulses, the body can act like a capacitor or virtual electrode sinking the current.

Secondly in FIG. 4, we provide an ultrasonic transducer 17, which can mechanically excite the needle-based sensor body and/or coating such as with acoustic waves 18. The transducer 17 is equipped with insulated electrical leads or connections such as 13a/13b to both power the transducer 17 and to sense the glucose sensor current generated by detection film 15. Again we see an adhesive pad 14a holding the sensor 1c needle upon the body 2c. The idea of the ultrasonic waves is that they can disrupt congealing foulant films and assure that the blood glucose can diffuse to the active chemical layer 15 such that it can electrochemically react and produce the desired detection electrical signal such as a current. It is a proven fact that ultrasound increases the permeability of polymers and gels to infiltrating mobile species and liquids. The electrical pulsing can assure that pathogenic fouling cellular matter can be killed or maimed perhaps such that it can also be more easily broken up by the ultrasonics. The ultrasonics can also extend the life of the sensor coating 15 as by assuring a permeable disrupted path is available to the sensor coating surface through and across any deposited foulants for glucose or other desired constituents to diffuse.

In all of our electrical-defouling and antipathogenic examples we remind the reader that the treatment voltages and currents may be extremely short, on the order of nanoseconds, to selectively alter or disrupt cell-interior features, somewhat longer such as in the microsecond to millisecond range to disrupt or rupture external cell membranes as by electroporation, or even continuous or CW in nature wherein one might electrophoretically move a mobile species as to pump it or filter it out of a liquid or flowstream, in addition to causing cellular damage. Continuous currents can also destroy and oxidize cellular constituents; however, they may require return electrodes or paths. Very short pulses can be delivered using capacitive virtual grounding as described with virtually no detectable temperature rise. Longer and particularly CW currents can generate temperature rises and can also beneficially kill pathogens. The pulsed electrical heating is also claimed on its own in addition to it or any type of heating being a secondary assisting mechanism. Longer or CW pulses may require the also-taught additional body electrode. This is quite similar to the case of RF based thermal ablation of human tissues wherein an electrode-based localized cutting wand delivers its current to a large-area body-applied electrode such as a pad the patient rests on. So if sustained currents are employed, a second electrode will likely act as a sink either on the device or on the body.

It will be recognized now that the invention is also quite suitable for defouling optical-based sensors. In addition to their infection or biological rejection and scarring possibilities, these sensors can become optically disrupted by even the thinnest of coatings such as biofilms or mineral deposits. The use of the electrical methods and ultrasound combined are quite attractive for this application.

For long rubbery or polymeric based catheters such as intrathecal catheters or long insulated wires or leads (e.g., electrodes for deep brain stimulation, for example) we believe that the electrical pulsing method is ideal. One could, for example, provide a surface conductive coating on the insulator or catheter rubber so that that surface and its nearby tissue/fluid environs could be pulse-cleaned occasionally or even constantly. For example, an insulated electrode insulated with a polymer could have that polymer carbon-infused on its surface such that the surface is a second electrode which is immune to flexural fatique.

Most of our above discussion addresses the electrical fields operating on cellular matter in, at or relatively close to the device with the pulsing capability. We once more mention that electrically altered cellular species could be circulated or diffused away from the device, as within a bloodstream, such that they provide remote (from the device) benefit. In that case, at least those treated or altered cells which move away from the device can be thought of as providing the device with an outreach capability beyond the intense field gradients or current densities.

The electrical method is also useful for avoiding or removing undesirable deposits on the internal surfaces of blood lumens and passages including veins, arteries, cardiac chambers and the aorta. The technique promises a means to preferably gradually do so so as not to create macroscopic problematic debris. One could insert an electrode wire into such an artery or vein temporarily or even long-term. By pulsing it on a schedule or in response to a sensed burden, one could provide a battery powered device for such lumen-cleaning therapy. Of course, our other complimentary mechanisms such as ultrasound, drugs, and UV could also be delivered along or within that electrode or coated upon the electrode or permeated into its surface. We anticipate an implantable system with a central pulser and multiple such electrodes being routed into one or multiple such veins or arteries. The pulsing should also keep the electrode itself clean and pathogen free. Such an electrode could fit loosely in the lumen (have a diameter smaller than the lumen) such that with body motion and perfusion it actually acts to scan nearby surfaces as they get close to the wire. The blood around the electrode acts to apply most of the voltage pulse potential-gradient across the vessel wall, including across any contaminants or plaques. If this is implemented correctly, then the patient will not even feel the wire system but will avoid the current cruder methods of such debris breakup and removal.

We have above taught the use of prepared or conditioned surfaces in combination with our two core antifouling mechanisms. One last class of conditioned surfaces would be surfaces having controlled surface charges. Such controlled electrical surface charges can be implemented in two basic ways: 1) By holding the surface at a potential as with an electrode, b) coating the surface with a film or charged species which inherently has a surface charge, at least when brought into contact with the foulant and/or aqueous environments.

Such charged surface materials may also be applied in layers which slowly detach or diffuse away to expose an underlying material or materials with a different charge as another means of preventing or controlling biofilm formation through the establishment of a polarity gradient. As an example, the outer surface could consist of an anionically charged coating of heparin which would gradually diffuse away over a period of hours or days, thereby exposing an inner coating which might consist of a strong positively charged material containing quaternary ammonium groups such as QAS or silver.

In another aspect of the invention, the medication or bioactive implant has the same charge as the surface or lumen of the article and is therefore variably repelled and released from the surface or lumen of the article either gradually or more quickly depending on the amount of voltage applied.

Examples of antimicrobial bioactive agents for ionic coupling or repulsion are vancomycin, gentamycin, and silver ion. For example, a catheter could be made to have a negative charge in order to attract vancomycin which is positively charged. Generally, the following categories of bioactive agents may be used: antibacterial agents or antimicrobial agents, anticoagulants, enzymes, hormones, vitamins, antibodies, growth factors, antimetabolites or antiproliferatives, antimitotics, vasodilators, vasospasm inhibitors, antihypertensives, antiplatelets, proteins, peptides, dyes, DNA and RNA segments, anti-cancer chemotherapeutic agents, anti-inflammatory steroids, analgesics, anesthetics, immunosuppressive agents, NSAIDs, free radical scavengers, radiotherapeutic agents, other heavy metal agents in addition to silver, or mixtures of any of these.

So, in summary, we have two core defouling/antifouling mechanisms of electrical pulses and employment of Laplacian orifices/membranes. They may be used together or separately. One or both may also be applied together with a host of listed known and future-anticipated defouling/antifouling mechanisms.

What is claimed is:

1. A method of inhibiting, avoiding or destroying existing, potential or incipient biodeposits, biofilms or pathogens any of (a) in or on a living body including in or on a medical device or implement placed, inserted or insertable in or on said body, (b) in, on or in contact with an ex vivo bodily tissue or fluid, or (c) in, on or in contact with a medium or matter to be consumed by, ingested by or exposed to a living being or entity, the method comprising at least one of:
i) application or inducement of an electrical field having any of a field-strength, polarity, pulse duration or frequency to at least one of cause (1) alteration or damage to cell-interior membranes or entities, (2) alteration or damage to cell-exterior membrane or membrane associated entities, (3) alteration or damage to intracellular or between-cell tissues or fluids, (4) cause a beneficial form of electrotransport or alteration of any natural or introduced species, (5) cause a beneficial damaging or altering current of an at least transient nature; or
ii) use of a Laplacian orifice or membrane wherein any one or more of: a) a flowable medium, constituent or species is pressure-switchable to a state of ingress, flow, non-ingress or non-flow, thereby allowing for an at least temporary dry or liquid-free state inhibitive of biofilms, biodeposits or pathogens, b) the orifice or membrane can be cleaned or defouled with the help of an ingressing or flowed flushing action from at least one direction, c) the orifice or membrane acts as an isolation valve resulting in avoidance or inhibition of fouling or pathogens in an isolated or isolatable region in communication with the orifice or membrane, d) the orifice or membrane is equipped with one or more electrodes of a man-made or virtual type which are employed to combat pathogens or fouling phenomenon in or at the orifice or membrane, e) the orifice or membrane is infused with a drug or species which beneficially treats a flowable medium passing through the orifice or membrane.

2. The method of claim 1 wherein at least one of said electric field application and Laplacian orifice application is combined with the use of at least one additional antifouling or antipathogen mechanism selected from the list of:
a) use of topographical surfaces which have an inherent antifouling topographical property;
b) use of a self-charging film or coating which presents a charged surface to or in cooperation with foulants or fouled mediums without the need to apply a voltage with an electrode;
c) use of a charged surface as by applying a potential with an electrode;
d) use of ultraviolet light or radiation;
e) use of ultrasound radiation for any of cleaning, heating, cavitation, streaming or sonoporation;
f) use of antifouling ions or complexes or radicals such as silver and copper based ions or radicals or ozone;
g) use of resistive or radiant heating;
h) use of unwettable or poorly wettable materials;
i) use of drugs, medicaments, chemical species or biologic materials which attack or kill foulants or pathogens as coatings or infusants on or in a material to be kept from fouling; and
j)) use of drugs, medicaments, chemical species or biologic materials which attack or kill foulants or pathogens as systemically or regionally delivered.

3. The method of claim 1 wherein any one or more of:
a) an electrical pulse duration or waveform can be measured in nanoseconds or less and/or an electrical field strength is between 0.01-1000 Kilovolts/cm and damage or alteration to at least one cell-interior entity or cell-interior membrane is caused, the damage or alteration being at least temporary in nature;
b) an electrical pulse duration or waveform is longer than nanoseconds and can be measured in microseconds, milliseconds or longer and/or an electrical field strength is between 0.01-100 Kilovolts/cm-and damage or beneficial alteration to a cell exterior membrane or constituent thereof is caused in at least one cell, said damage or alteration being at least temporary in nature;
c) an electrical pulse duration or waveform causes damage or alteration to a tissue or body fluid portion surrounding or outside of at least one cell;
d) an electrical field or electrical current causes a beneficial form of electrotransport of a natural or artificially introduced species or of a charge-carrier; or
e) said damage, alteration or electrotransport takes place in or on any of a living subject, a subjects body fluid or tissue whether in vivo or ex vivo, in or on a medical device or in or on an equipment or processing apparatus potentially prone to fouling.

4. The method of claim 3 wherein apoptosis, necrosis or any form of cell death or destruction is caused, triggered or more likely in any one or more cells or wherein any affected cell(s) or entities has or have diminished or altered viability.

5. The method of claim 3 wherein an at least temporary electrical field is accompanied by an at least momentary or transient current, at least one of an applied or induced voltage or applied or induced current having resulted in, induced or caused said electric field.

6. The method of claim 3 wherein either or both of an applied or induced voltage or applied or induced current are any of measured, sensed, detected, controlled, patient-customized, limited for any reason including safety, delivered with a duty-cycle, repeated or adjusted or set for a particular electrode.

7. The method of claim 1 wherein a permeable orifice or membrane, Laplacian or not, is equipped with one or more electrodes, the electrode(s) being utilized for at least one of: i) application of electric field or current pulses or waveforms which cause cell-internal damage or alteration of biological constituents therein or thereon the internal entities, ii) application of electric field or current pulses or waveforms which cause cell-external membrane damage or alteration of biological constituents therein or thereon the external membrane(s), iii) application of electrical current pulses or waveforms which damage or alter cells or constituents therein, thereon or juxtaposed thereto, iv) electrical or electrically-assisted transport, pumping, filtering or component separation of a medium or constituent in, passing-into or passing-through the orifice or membrane, or (v) treating of any portion of the orifice or membrane itself and/or of any actual or potential fouling therein or thereon and/or treating of orifice/membrane passed, passing, passable or adjacent constituents or tissues/fluids, vi) electrowetting, electroosmosis or electrophoretic driving or switching of a fluid or mobile species.

8. The method of claim 1 wherein an electric field or current is applied or induced for any patient-beneficial purpose any one or more of:
i) using an electrically conductive, ionically conductive, photoconductive or semiconductive flowable medium or liquid to communicate, allow passage-of, allow sinking-of or to apply any of a voltage or current;
ii) using a man-made electrode of any design or material including conducting, photoconducting, or semiconductive wires, electrode films, electrode thinfilms, electrode plates or meshes, electrical transmission lines, needles, catheters, clips or clamps, or using any other conductive medical device or member thereof;
iii) utilizing a subject or patients tissue, body fluid or anatomy capacitance in a manner wherein it can be considered a circuit element, virtual electrode, virtual current sink and/or wherein it can be considered advantageous such as by avoiding the use of one or more physical man-made electrodes;
iv) utilizing a man-made conducting, photoconducting or semiconducting electrode or plate that is either in direct contact with tissue/fluid or that is spaced from said tissue/fluid with a capacitive layer;
v) using a high voltage pulse generator and/or high voltage switch or trigger;
vi) using, applying or inducing at least one electric field or current or waveform that is any one or more of a bipolar, unipolar, DC, DC-pulsed, AC, AC-pulsed, pulsed in any manner, time-varying, CW or continuous-wave, multiwaveform, harmonic in nature, sinusoidal, square wave, ramped wave, sawtooth wave or any waveform which alters or kills cells or causes beneficial electrotransport, destruction, damage or alteration in a desirable manner;
vii) utilizing an applied or induced bias voltage or bias current in any combination with a pulsed voltage or current regardless of relative timing;
viii) utilizing or inducing a voltage or current or waveform that is selected for a specific patient, specific foulable implement or device, for a specific state of actual or potential fouling or specific pathogen presence or for a specific electrotransport application;
ix) utilizing any cylindrical, elongated, wire-like, transmission-line, film-like, or shaped electrode or any electrode situated on any tip, edge, face or internal/external surface of an invasive or non-invasive medical impalement or device-by electrode meaning man-made conducting, semiconducting, photoconducting material electrodes as well as conductive flowable media or virtual electrodes or current sinks;
x) using one or more man-made or virtual electrodes or current sinks arranged in one, two or three dimensions or arranged in an array or pattern, said electrodes which may or may not simultaneously operate or apply, induce or sink the identical voltage or current waveforms; or
xi) using a foulable implement or foulable device wherein the implement or device provides at least a first electrode and an at least second electrode is provided but is not necessarily itself also mounted on or in the implement or device, the at least two electrodes each individually being at least one of (a) a man-made electrode, (b) a conductive flowable medium electrode or (c) a virtual electrode formed by the capacitance of a treated device or by a tissue capacitance.

9. The method of claim 1 wherein the device, implement, bodily tissue or fluid, medium or matter being or being kept defouled or free of unacceptable pathogens is or has any one or more of:
i) is a man-made catheter, scope, lumen, stent or graft regardless of whether it is made of engineering material, engineered biomaterial or of formerly living tissue;
ii) is a drug or nourishment delivery catheter, lumen, needle or trocar;
iii) is any type of drainage catheter, lumen, needle or trocar including intrathecal catheters and urine drainage catheters;
iv) is any type of implanted medical device or component thereof, whether said implantation is short-term or long-term;
v) is an electrical lead or optical fiber supporting or acting as a medical therapy or surgery sensor or device such as a deep-brain stimulation electrode;
vi) has one or more antifouling, defouling or antipathogenic electrodes passed into or through it, mounted upon it or serving as a device or implement component;
vii) has its electrical defouling or antipathogenic behavior powered by a battery or fuel cell;
viii) is a sensor or a wound-care device, implement, bandage or closure;
ix) is an artificial limb, prosthesis or medical implant;
x) is a body fluid, tissue or waste material;
xi) is a material or substance which will potentially be or is ingested, eaten, drunk, breathed, inhaled by or worn by a living being or otherwise brought into contact with a living being;
xii) is a substance being antifouling or antipathogenically treated that is flowed or passed through or past the treating entity at least once such that some substance arriving from remote or treatment-remote locations can be treated, said substance optionally being recirculated for two or more such treatments;
xiii) is a natural or artificial bodily lumen, a placed lumen-associated stent, graft or intraluminal device, or a wound, any of which is subject to any type of sclerotic, mineral or fat deposits or any other reactive deposit, infection or inflammation;
xiv) is bodily matter subject to damage, stress or destruction due to a neurodegenerative disease;
xv) is an engineered bodily fluid substitute such as a blood replacement fluid or CSF replacement fluid, said replacement being at least for a short period, said replacement optionally involving dilution of the natural bodily fluid rather than its complete removal; or
xvi) is a transplanted, donated or transfused organ, tissue or body fluid regardless of origin.

10. The method of claim 1 wherein the fouling deposits are deposits on the inside of passages, including blood filled lumens, vasculature, arteries, cardiac chambers or the aorta and the method is applied to avoid, reduce or remove at least some of said deposits, an electrical field or current preferably being applied with the help of a man-made electrode or liquid-based electrode inside said lumen, said liquid possibly including blood or conduction-enhanced blood, the man-made or liquid electrode being temporarily or permanently placed.

11. A method of inhibiting, avoiding or destroying existing, potential or incipient biodepo sits, biofilms or pathogens any of (a) in or on a living body including in or on a medical device or implement placed, inserted or insertable in said body, (b) in, on or in contact with an ex vivo bodily tissue or fluid, or (c) in, on or in contact with a medium or matter to be consumed, ingested by or exposed to a living being or entity, the method comprising at least one of:
i) application or inducement of an electrical field having any of a field-strength, polarity, pulse duration or frequency necessary to at least one of cause (1) alteration of damage to cell-interior membranes or entities, (2) alteration of damage to cell-exterior membrane or membrane associated entities, (3) alteration or damage to intracellular or between-cell tissues or fluids, (4) cause a beneficial form of electrotransport or alteration of any natural or introduced species, (5) cause a beneficially damaging or altering current of an at least transient nature; or
ii) use of a Laplacian orifice or membrane wherein any one or more of: a) a flowable medium, constituent or species is pressure-switchable to a state of ingress, flow, non-ingress or non-flow, thereby allowing for an at least temporary dry or liquid-free state inhibitive of biofilms, biodeposits or pathogens, b) the orifice or membrane can be cleaned or defouled with the help of an ingressing or flowed flushing action from at least one direction, c) the orifice or membrane acts as an isolation valve resulting in avoidance or inhibition of fouling or pathogens in an isolated or isolatable region in communication with the orifice or membrane, d) the orifice or membrane is equipped with one or more electrodes of a man-made or virtual type which are employed to combat pathogens or fouling phenomenon in, at or on the orifice or membrane and
combined with the additive or synergistic simultaneous, sequential or interleaved application of at least one of the additional antifouling, defouling or antipathogenic mechanisms of:
a) use of applied acoustical or ultrasonic energy, said intensity or energy level causing one or more of prevention, removal or destruction of fouling entities and/or pathogenic entities, said energy possibly being any one or more of noncavitating, cavitating, nonthermal, thermal, acoustically-streaming, able to cause thermal effects such as thermal necrosis of cells, or able to cause sonoporation, transfection, cell-destruction or cell fusion;
b) use of applied ultraviolet light or UV to prevent, remove or destroy any of fouling entities and/or pathogenic entities;
c) use of engineered surface properties on in situ surfaces, including the use of biofilm inhibiting surface topography, surface-tensions, surface charge-states/polarizations such as provided by surface conditioning, surface-activation, surface polarization, surface-coating, surface-composition control or manipulation of the material's or surface's inherent electronegativity to prevent, remove or destroy any of fouling entities of pathogenic entities;
d) use of in situ static or pseudostatic pressurization or shock events to promote unplugging, defouling or flushing;
e) use of an in situ antipathogenic or antifouling drug, chemical or radiation exposure, including any gaseous, plasma, liquid, semisolid or solid-state ionic exposure, including exposure to silver or copper ions or ozone, or exposure to damaging or ionizing radiation, including ionizing or nuclear radiation or nuclear particle fluxes;
f) use of in situ heating or freezing; or
g) use of in situ electrical or ion currents wherein an electrical current is caused or maintained for a useful defouling or antipathogenic period, the current formed by natural and/or artificial charge carriers or ions.

12. The method of claim 11 wherein any one or more of:
a) an electrical pulse duration or waveform can be measured in nanoseconds or less and/or an electrical field strength is between 0.01-1000 Kilovolts/cm and damage or alteration to at least one cell-interior entity or cell-interior membrane is caused, the damage or alteration being at least temporary in nature;
b) an electrical pulse duration or waveform is longer than nanoseconds and can be measured in microseconds, milliseconds or longer and/or an electrical field strength is between 0.01-100 Kilovolts/cm-and damage or beneficial alteration to a cell exterior membrane or constituent thereof is caused in at least one cell, said damage or alteration being at least temporary in nature;
c) an electrical pulse duration or waveform causes damage or alteration to a tissue or body fluid portion surrounding or outside of at least one cell;
d) an electrical field or electrical current causes a beneficial form of electrotransport of a natural or artificially introduced species or of a charge-carrier; or
e) said damage, alteration or electrotransport takes place in or on any of a living subject, a subjects body fluid or tissue, whether in vivo or ex vivo, in or on a medical device or in or on an equipment or processing apparatus potentially prone to fouling.

13. The method of claim 12 wherein apoptosis, necrosis or any form of cell death or destruction is caused, triggered or more likely in any one or more cells or wherein any affected cell(s) or entities has or have diminished or altered viability.

14. The method of claim 12 wherein an at least temporary electrical field is accompanied by an at least momentary or transient current, at least one of an applied or induced voltage or applied or induced current having resulted in, induced or caused said electric field.

15. The method of claim 12 wherein either or both of an applied or induced voltage or applied or induced current are any of measured, sensed, detected, controlled, patient-customized, limited for any reason including safety, delivered with a duty-cycle, repeated or adjusted or set for a particular electrode.

16. The method of claim 11 wherein a permeable orifice or membrane, Laplacian or not, is equipped with one or more electrodes, the electrode(s) being utilized for at least one of: i) application of electric field or current pulses or waveforms that cause cell-internal damage or alteration of biological constituents therein or thereon the internal entities, ii) application of electric field or current pulses or waveforms which cause cell-external membrane damage or alteration of biological constituents therein or thereon the external membrane(s), iii) application of electrical current pulses or waveforms which damage or alter cells or constituents therein, thereon or juxtaposed thereto, iv) electrical or electrically-assisted transport, pumping, filtering or component separation of a medium or constituent in, passing-into or passing-through the orifice or membrane, v) treating of any portion of the orifice or membrane itself and/or of any actual or potential fouling therein or thereon and/or treating of orifice/membrane passed, passing, passable or adjacent constituents or tissues/fluids, or vi) electrowetting, electroosmosis or electrophoretic driving or switching of a fluid or mobile species.

17. The method of claim 11 wherein the device, implement, bodily tissue or fluid, medium or matter being or being-kept defouled or free of unacceptable pathogens is any one or more of:
i) a man-made catheter, lumen, stent or graft regardless of whether it is made of engineering material, engineered biomaterial or formerly living tissue;
ii) a drug or nourishment delivery catheter, lumen, needle or trocar;
iii) any type of drainage catheter, lumen, needle or trocar including intrathecal catheters and urine drainage catheters;
iv) any type of implanted medical device, whether said implantation is short-term or long-term;
v) an electrical lead or optical fiber supporting or acting as a medical therapy or surgery sensor or device such as a deep-brain stimulation electrode;
vi) has one or more antifouling, defouling or antipathogenic electrodes passed into or through it, mounted upon it or serving as a device or implement component;
vii) has its electrical defouling or antipathogenic behavior powered by a battery or fuel cell;
viii) is a sensor or a wound-care device, implement, bandage or closure;
ix) an artificial limb or prosthesis;
x) a body fluid, tissue or waste material;
xi) a material or substance ingested, eaten, drunk, breathed, inhaled by or worn by a living being or otherwise brought into contact with a living being;
xii) a substance being antifouling or antipathogenically treated that is flowed or passed through or past the treating entity at least once such that some substance arriving from remote or treatment-remote locations can be treated, said substance optionally being recirculated for two or more such treatments;
xiii) a natural or artificial bodily lumen, a placed lumen-associated stent, graft or intraluminal device, or a wound, any of which is subject to any type of sclerotic, mineral or fat deposits or any other reactive deposit, infection or inflammation;
xiv) bodily matter subject to damage, stress or destruction due to a neurodegenerative disease;
xv) an engineered bodily fluid substitute such as a blood replacement fluid or CSF replacement fluid, said replacement being at least for a short period, said replacement optionally involving dilution of the natural bodily fluid rather than its complete removal; or
xvi) a transplanted, donated or transfused organ, tissue or body fluid regardless of origin.

18. The method of claim 11 wherein an electric field or current is applied or induced for any patient-beneficial purpose any one or more of:
i) using an electrically conductive, ionically conductive, photoconductive or semiconductive flowable medium or liquid to communicate, allow passage of, allow sinking of or to apply any of a voltage or current;
ii) using a man-made electrode of any design or material, including conducting, photoconducting, or semiconductive wires, electrode films, electrode thinfilms, electrode plates or meshes, electrical transmission lines, needles, catheters, clips or clamps, or using any other conductive medical device or member thereof;
iii) utilizing a subject's or patient's tissue, body fluid or anatomy capacitance in a manner wherein it can be considered a circuit element, virtual electrode, virtual current sink and/or wherein it can be considered advantageous such as by avoiding the use of one or more physical man-made electrodes;
iv) utilizing a man-made conducting, photoconducting or semiconducting electrode or plate that is either in direct contact with tissue/fluid or that is spaced from said tissue/fluid with a capacitive layer;
v) using a high voltage pulse generator and/or high voltage switch or trigger;
vi) using, applying or inducing at least one electric field or current or waveform that is any one or more of a bipolar, unipolar, DC, DC-pulsed, AC, AC-pulsed, pulsed in any manner, time-varying, CW or continuous-wave, multiwaveform, harmonic in nature, sinusoidal, square wave, ramped wave, sawtooth wave or any waveform which alters or kills cells or causes beneficial electrotransport, destruction, damage or alteration in a desirable manner;
vii) utilizing an applied or induced bias voltage or bias current in any combination with a pulsed voltage or current regardless of relative timing;
viii) utilizing or inducing a voltage or current or waveform that is selected for a specific patient, specific foulable implement or device, for a specific state of actual or potential fouling or specific pathogen presence or for a specific electrotransport application;
ix) utilizing any cylindrical, elongated, wire-like, transmission-line, film-like, or shaped electrode or any electrode situated on any tip, edge, face or internal/external surface of an invasive or non-invasive medical impalement or device-by electrode meaning man-made conducting, semiconducting, photoconducting material electrodes as well as conductive flowable media or virtual electrodes or current sinks;
x) using one or more man-made or virtual electrodes or current sinks arranged in one, two or three dimensions or arranged in an array or pattern, said electrodes which may or may not simultaneously operate or apply, induce or sink the identical voltage or current waveforms; or
xi) using a foulable implement or foulable device wherein the implement or device provides at least a first electrode and an at least second electrode is provided but is not necessarily itself also mounted on or in the implement or device, the at least two electrodes each individually being at least one of (a) a man-made electrode, (b) a conductive flowable medium electrode or (c) a virtual electrode formed by the capacitance of a treated device or by a tissue capacitance.

19. A medical device, implement or prosthesis that can be kept in a safe defouled, pathogen-free or pathogen-reduced condition with the help of an applied or induced electric field or fields comprising:
a) an invasive or non-invasive medical device, patient treatment implement or prosthesis that serves a beneficial medical or physiological purpose but which may be subject to or cause potential fouling or pathogen development in association with its use or presence;
b) a high voltage-gradient application circuit and one or more leads and application electrodes coupled to the device, implement or prosthesis such that a high voltage gradient can be applied to at least some portion of the device, implement or prosthesis and/or to some portion of a patients or subjects anatomy, tissue or fluids in any one or more of an in vivo, ex vivo, invasive or non-invasive manner;
c) the high voltage gradient being applied or induced with a voltage or current waveform to at least temporarily beneficially alter or destroy at least one of a cell interior membrane or entity or cell exterior membrane or entity;
d) said beneficial altering or destruction contributing to or providing an antifouling, defouling or antipathogenic ability;
e) the device, implement or prosthesis containing, providing, supporting, integrating or controlling at least one said application electrode, said electrode(s) being at least one of man-made, utilizing a flowable conductive medium, utilizing a photoconductor or semiconductor, being an electrical transmission line, or being a virtual electrode utilizing a body or device capacitance; and
f) the at least one electrode being conductively coupled or capacitively coupled to a treated device, implement, prosthesis, anatomy, tissue or fluid subject to actual or potential fouling.

20. The device, implement or prosthesis of claim 19 wherein any of:
i) at least one application electrode is a man-made metal-containing electrode;
ii) an electrical pulse or individual waveform or waveform transition has a period in the nanosecond or nanoseconds regime or shorter;
iii) an electrical pulse or individual waveform or waveform transition has a period in the microseconds or milliseconds regimes or longer;
iv) at least one electrode is any one or more of extended in one, two or three dimensions, is wrapped around a surface, is situated on or at an end, edge, tip, exterior surface, or interior surface of a device, implement, prosthesis or tissue, or is inserted into a living body in any manner;
v) the device, implement or prosthesis is non-invasive, minimally-invasive, invasive, implanted or worn on or coupled-to the body;
vi) an electrical current generated by a pulse(s), waveform or waveform transition in or on the device, implement, prosthesis or bodily matter is magnitude-limited or temporally-limited to be safe for a patient, or subject;
vii) an electrical current is any of controlled, monitored, limited, avoided or part of a treatment control feedback loop or monitoring component;
viii) the device, implement or prosthesis is a sensor such as an implanted glucose sensor or blood flow sensor;
ix) the device or implement is a catheter, needle or other man-made lumen utilized for drug or nutrition delivery or for any type of drainage or pressure-control;
x) the device, implement or prosthesis includes a wired or wireless electrical or optical communication port or interface;
xi) an applied or induced field is applied or induced utilizing any of a man-made electrode, a liquid-conductive electrode or a capacitive virtual electrode;
xii) an applied or induced field is applied or induced between at least two electrodes of any type including two man-made electrodes; or
xiii) the device or implement has its own electrode for another purpose and that electrode is shared.

21. The medical device, implement or prosthesis of claim 19 wherein the device, implement, prosthesis, anatomy, tissue or fluid being or being-kept defouled or free of unacceptable pathogens is any one or more of:
i) a man-made catheter, lumen, scope, stent or graft regardless of whether it is made of engineering material, engineered biomaterial or formerly living tissue;
ii) a drug or nourishment delivery catheter, lumen, needle or trocar;
iii) any type of drainage catheter, lumen, needle or trocar including intrathecal catheters and urine drainage catheters;
iv) any type of implanted medical device, whether said implantation is short-term or long-term;
v) an electrical lead or optical fiber supporting or acting as a medical therapy or surgery sensor or device such as a deep-brain stimulation electrode;
vi) has one or more antifouling, defouling or antipathogenic electrodes passed into or through it, mounted upon it or serving as a device or implement component;
vii) has its electrical defouling or antipathogenic behavior powered by a battery or fuel cell;
viii) is a sensor or a wound-care device, implement, bandage or closure;
ix) an artificial limb or prosthesis;
x) a body fluid, tissue or waste material;
xi) a material or substance ingested, eaten, drunk, breathed, inhaled by or worn by a living being or otherwise brought into contact with a living being;
xii) a substance being antifouling or antipathogenically treated that is flowed or passed through or past the treating entity at least once such that some substance arriving from remote or treatment-remote locations can be treated, said substance optionally being recirculated for two or more such treatments;
xiii) a natural or artificial bodily lumen, a placed lumen-associated stent, graft or intraluminal device, or a wound, any of which is subject to any type of sclerotic, mineral or fat deposits or any other reactive deposit, infection or inflammation;
xiv) bodily matter subject to damage, stress or destruction due to a neurodegenerative disease;
xv) an engineered bodily fluid substitute such as a blood replacement fluid or CSF replacement fluid, said replacement being at least for a short period, said replacement optionally involving dilution of the natural bodily fluid rather than its complete removal; or
xvi) a transplanted, donated or transfused organ, tissue or body fluid regardless of origin.

22. A medical or industrial device, implement, equipment-article or storage/handling-entity that can be kept in a safely defouled, pathogen-free or pathogen-reduced condition with the help of a membrane or orifice which has one or more of the antifouling or antipathogenic attributes of:
a) a Laplacian orifice or membrane which can be pressure switched at least once between a wetted or ingressed state and a less wetted, ingressed or dry state, thereby inhibiting at least one of orifice or membrane internal or surface fouling or pathogenicity, said ingress or wetting being that of at least one constituent or species, said ingress, wetting, egress or dewetting pressure being applied in any fluidic, pneumatic, vacuum, electrically-aided or acoustical-streaming way;
b) any orifice or membrane, whether Laplacian or not, that has or is equipped with or juxtaposed with one or more man-made electrodes, virtual electrodes utilizing conductive fluid, or any electrode using a capacitive current sinking effect, the electrode(s) being utilized to at least one of alter, damage or transport species or cells in, at or near said orifice or membrane, said alteration, damage or transport contributing to the antifouling or antipathogenic capability; and
c) the orifice or membrane of (b) wherein one or more electrodes is utilized to treat any one or more of species, cells or constituents passing through, residing upon or residing in the orifice or membrane;
the orifice or membrane being part of or coupled to the entity in a manner reducing or avoiding fouling or pathogenic development therein, thereon or in a substance processed, flowed or stored in or through said entity.

23. The medical or industrial device, implement, equipment-article or storage/handling entity of claim 22 being kept defouled or free of unacceptable pathogens wherein it is any one or more of:
i) a man-made catheter, lumen, stent or graft regardless of whether it is made of engineering material, engineered biomaterial or formerly living tissue;
ii) a drug or nourishment delivery catheter, lumen, needle or trocar;
iii) any type of drainage catheter, lumen, needle or trocar including intrathecal catheters and urine drainage catheters;
iv) any type of implanted medical device-whether said implantation is short-term or long-term;
v) an electrical lead or optical fiber supporting or acting as a medical therapy or surgery sensor or device, including a deep-brain stimulation electrode;
vi) has one or more antifouling, defouling or antipathogenic electrodes passed into or through it, mounted upon it or serving as a device or implement component;
vii) has its electrical defouling or antipathogenic behavior powered by a battery or fuel cell;
viii) is a sensor or a wound-care device, implement, bandage or closure;
ix) an artificial limb or prosthesis;
x) a body fluid, tissue or waste material;
xi) a material or substance ingested, eaten, drunk, breathed, inhaled by or worn by a living being or otherwise brought into contact with a living being; or
xii) a food, water, beverage, drug, biological-matter, medicament or breathable gas storage, delivery or processing pipeline, duct, tank, valve or reservoir including such storage, delivery, processing or transport in product packaging, product-containers or product transport as in vehicles of any sort.

24. A device or implement for treating cancer or other cellular-affecting disease or disorder, the device or implement being any one or more of non-invasive, minimally invasive, invasive, tissue-puncturing or implanted and used any one or more of in vivo or ex vivo, the device or implement including at least one electrode capable of applying or inducing an electric field to beneficially alter or kill at least some diseased cells, the applied or induced electric field being applied with one or both of pulsed waveforms or pulsed wave transitions short enough to alter, damage, render unviable or kill entities within one or more cells and pulsed or continuous waveforms or waveform transitions long enough to alter, damage, render unviable or kill the outer membrane or membrane associated entities of one or more cells.

25. The device or implement of claim 24 wherein any one or more of: i) cancerous cells themselves have one or both of at least some of their internal contents or external membranes or constituents thereof at least temporarily altered or destroyed, ii) alteration or destruction of a first cell-type internal or external cell portion or entity itself directly or indirectly causes biological alteration or destruction of a second cell-type internal or external portion or entity contributing to cancer treatment-none, one or both cell types being cancerous cells, iii) a patient or subject-beneficial electrotransport of a natural or man-made mobile constituent or species also takes place whether or not simultaneously.

26. The device or implement of claim 24 wherein any of a device, implement, body tissue or body-fluid being protected is any one or more of:
i) a bodily organ such as a brain, gland, pancreas, liver or bladder, whether in vivo or ex vivo at the time of treatment;
ii) a bodily member or limb, including a head, leg, arm, foot or breast, whether or not part of a complete living or nonliving being at the time of treatment;
iii) any portion of a living being historically or genetically prone to cancer, whether in vivo or ex vivo at the time of treatment;
iv) a bodily fluid, including blood or CSF, whether in vivo or ex vivo at the time of treatment;
v) a medical implant whether it is fabricated from engineering materials, natural materials or living or once-living cells or tissues, whether in vivo or ex vivo at the time of treatment;
vi) a living being potentially subject to or having been the subject of biological or germ warfare agents;
vii) a living being otherwise potentially or actually subject to a disease or one of its undesirable symptoms;
viii) an entire living body;
ix) two or more living bodies wherein treatment is sequential or simultaneous;
x) a person who is known to handle food or drink which might become contaminated by the person;
xi) a doctor, dentist or health-practitioner who wishes not to transfer a disease or illness from patient to patient or between himself/herself and a patient; or
xii) a patient whose obesity disorder is treated to reduce or avoid at least some fat deposits.

27. A medical device, device component, tool, implant or implement which comes into contact with a bodily tissue or fluid and thereby may become fouled, the device having an antifouling mechanism utilizing a pulsed electric field comprising:
a medical device, device component, tool, implant or implement subject to actual or potential fouling;
a generator or electrical energy source or reservoir for generating or providing an electrical pulse; and
a router for routing the pulse to at least a portion of the medical device needing defouling or antifouling;
the pulse being discharged or routed through at least some tissue, bodily fluid or foulant needing antifouling treatment.

28. The device of 27 wherein a pulse or waveform is a nanosecond or nanoseconds length pulse and it damages or destroys cell-interior membranes or vesicles.

29. The device of 27 wherein a pulse or waveform is on the order of microseconds to milliseconds long and it damages or destroys cell-exterior membranes or vesicles.

30. The device of 27 wherein the pulse or waveform is long enough or intense enough to cause thermal heating and necrosis cell death.

31. The device of 27 wherein an electric field is also applied for the purpose of iontophoretically or electrophoretically transporting a drug, medicament or other tissue or fluid mobile species.

32. The device of 27 wherein a single manmade electrode is utilized and a second electrode is provided by a virtual electrode formed by a tissue or bodily-fluid capacitance.

33. The device of 27 wherein two or more man-made electrodes are provided.

34. The device of 27 wherein a pulsed electric field is applied across or within a permeable orifice or membrane through which a flowable medicament, drug, bodily fluid passes or is passed, the orifice or membrane possibly requiring Laplacian activation pressure to ingress it.

35. The device of claim 27 wherein at least one electrode is employed comprising: a) a metal containing electrode, b) a man-made electrode, c) an electrode that employs ionic conduction, d) an electrode that employs an ionically conductive flowable medium, e) an electrode that employs an ionically conducting drug, medicament, water-solution or saline, f) a patterned electrode, g) a platinum or gold electrode, h) a wire electrode, i) a transmission-line electrode, or j) any electrode connected to a transmission line.

36. The device of claim 27 wherein a source of pulsed electrical energy utilizes at least one of: a) a spark discharge phenomenon, or b) one or more coils.

37. The device of claim 27 wherein pulsing is done at least one of: a) continuously, b) on a duty cycle, c) occasionally, or d) in response to a state of fouling.

38. A lumen-based medical device which passes or has passed a flowed medium through said lumen for its proper functioning, the device utilizing a fouling avoidance or fouling removal mechanism employing a Laplacian orifice or membrane comprising:
a lumen-containing medical device wherein a flowable medium or fluid is to be passed, at least once, through said lumen in at least one direction; and
a Laplacian orifice or membrane placed in a flowpath of said lumen and able to enable or disable said flow and/or filling of said lumen with the medium as by adjustment of the Laplacian pressure experienced by the orifice or membrane,
the device operable in two states, a flowing or filled state which is subject to fouling and an unflowing or unfilled state which is more resistant or impervious to fouling.

39. The device of claim 38 wherein any one or more of:
a) the device is a drainage, pressure-control or pressure measurement catheter;
b) the device is a delivery catheter;
c) the orifice or membrane is flow-activated by a positive pressure such as urine pressure;
d) the orifice or membrane is flow activated by a negative pressure such as a suction;
e) the orifice or membrane also acts to expose flowed medium to an electrical field which kills or suppresses foulants; or
f) the orifice or membrane also acts to expose flowed medium to a drug, medicament or antipathogenic species.

40. A device or system which is protected from fouling using a combination of:
a) at least one pulsed electric field to damage or kill an undesirable viable species or viable cells and/or at least one flow-switching Laplacian orifice or membrane which can isolate at least one device or system portion from fouling by time-limiting or avoiding immersion or wetting in a flowable medium causing fouling; and
b) at least one statically or pseudostatically charged surface, said charged surface serving to repel or prevent agglomeration or interfering with the survival of attaching or depositing foulants,
wherein said charged surface is charged or caused to be charged for at least a period using any one or more of an applied potential, a potential induced by surface-exposedcomposition, a potential induced by photoconductive mechanisms or a potential established by the above electrical pulsing means.

41. The device or system of claim 40 wherein at least one of said electric field application and Laplacian orifice application is combined with the use of at least one additional antifouling or antipathogen mechanism from the list of:
a) use of topographical surfaces which have an inherent antifouling topographical property;
b) use of a self-charging film or coating which presents a charged surface to foulants without the need to apply a voltage with an electrode;
c) use of a charged surface as by applying a potential with an electrode;
d) use of ultraviolet light or radiation;
e) use of ultrasound radiation for any of cleaning, heating, cavitation, streaming or sonoporation;
f) use of antifouling ions or complexes or radicals such as silver and copper based ions or radicals or ozone;
g) use of resistive or radiant heating;
h) use of unwettable or poorly wettable materials;
i) use of drugs, medicaments, chemical species or biologic materials which attack or kill foulants or pathogens as coatings or infusants on or in a material to be kept from fouling; and
j)) use of drugs, medicaments, chemical species or biologic materials which attack or kill foulants or pathogens as systemically delivered or regionally.
